(19) United States
(12) Patent Application Publication
(10) Pub. No.: US 2010/0210732 A1
(43) Pub. Date: Aug. 19, 2010

(54) Methods of Preventing the Serotonin Syndrome and Compositions for Use Therefor

(76) Inventor: Najib Babul, Blue Bell, PA (US)

Correspondence Address: Najib Babul, 146 Medinah Drive, Blue Bell, PA, 19422-3212, US

(21) Appl. No.: 12/223,987

(22) PCT Filed: Nov. 2, 2006
(86) PCT No.: PCT/US2006/042962

§ 371 Date: Aug. 13, 2008

Related U.S. Application Data
(60) Provisional application No. 60/732,121, filed on Nov. 2, 2005.

(51) Int. Cl. A61K 31/135; A61P 25/00

(52) U.S. Cl. 514/646

(57) ABSTRACT

The present invention is directed to pharmaceutical compositions and the use thereof for preventing or minimizing the intensity of the serotonin syndrome. The present invention is directed at a method of preventing or minimizing the intensity of the serotonin syndrome in humans which comprises administering proserotonergic agents and serotonin surge protectors, wherein said concurrent administration reduces or prevents serotonin excess, which is the cause of the serotonin syndrome. The present invention is also directed to pharmaceutical compositions comprising proserotonergic agents and serotonin surge protectors useful for carrying out the method of the present invention.

The application claims the benefit of U.S. Provisional Application No. 60/732,121, filed Nov. 2, 2005, which is herein incorporated by reference in its entirety.

### FIELD OF THE INVENTION

The present invention is in the field of pharmaceutical compositions and the use thereof for preventing or minimizing the risk of the serotonin syndrome.

### BACKGROUND ART

The serotonin syndrome is a potentially life-threatening adverse drug experience that results from therapeutic drug use, intentional self-poisoning or inadvertent interactions between drugs. Several salient features of the serotonin syndrome are critical to an understanding of the disorder. First, the syndrome is not an idiopathic iatrogenic reaction; it is a predictable consequence of serotonin excess in both the central and peripheral nervous systems. Second, excess serotonin agonism produces a wide constellation of clinical findings. Third, manifestations of the serotonin syndrome range from mild to fatal. (Boyer and Shannon, NEJM, 2005; Jones and Story, Anaesth Intensive Care, 2005; Sporer, Drug Safety, 1995).

This syndrome is characterized by a constellation of symptoms. Patients with mild cases may be afebrile but have tachycardia, shivering, diaphoresis, or mydriasis. The neurologic examination may reveal intermittent tremor or myoclonus, as well as hyperreflexia. In moderate cases of the serotonin syndrome, patients may have tachycardia, hypertension, and hyperthermia. A core temperature of up to 40° C. is common in moderate intoxication. Common features include mydriasis, hyperreflexia, clonus, hyperactive bowel sounds, diaphoresis, mild agitation or hypervigilance, as well as pressured speech. Patients with severe cases of the serotonin syndrome may have severe hypertension and tachycardia that may progress to frank shock. Such patients may have delirium, muscular rigidity and hypertonicity. Although no laboratory tests confirm the diagnosis of the serotonin syndrome, lab abnormalities that occur in severe cases include metabolic acidosis, rhabdomyolysis, elevated levels of AST, ALT and creatinine, seizures, renal failure, and disseminated intravascular coagulopathy (DIC). (Boyer and Shannon, NEJM, 2005; Jones and Story, Anaesth Intensive Care, 2005; Sporer, Drug Safety, 1995).

The incidence of the serotonin syndrome appears to have increased with the widespread use of drugs that enhance the effects of serotonin, usually through reuptake inhibition or direct agonism (“proserotonergic agents”). A wide variety of proserotonergic drugs, taken alone or in combination have been implicated in the causation of the serotonin syndrome (Boyer and Shannon, NEJM, 2005; Jones and Story, Anaesth Intensive Care, 2005; Sporer, Drug Safety, 1995). These include the now ubiquitous selective serotonin-reuptake inhibitors (SSRIs), e.g., citalopram, fluoxetine, fluvoxamine, paroxetine and sertaline; selective serotonin-norepinephrine reuptake inhibitors (SNRIs), e.g, venlafaxine, milnacipran; tricyclic and non-tricyclic antidepressants, e.g., buspirone, clomipramine, nefazodone, trazadone; monoamine oxidase (MAO) inhibitors, e.g., clorgiline, isocarboxazid, moclobemide, phenelzine and selegiline; antiepileptics, e.g., valproate; analgesics, e.g., fentanyl, meperidine, pentazocine and tramadol; antiemetic agents, e.g., granisetron, metoclopramide and ondansetron; antimigraine drugs, e.g., sumatriptan; bariatric medications, e.g., sibutramine; antibiotics, e.g., linezolide (a MAOI) and ritonavir (via CYP-450 3A4 inhibition); antitussives, e.g. dextromethorphan; dietary supplements and herbal products, e.g., tryptophan, Hypericum perforatum (St. John's wort), Panax ginseng (ginseng); and lithium. (Boyer and Shannon, NEJM, 2005; Jones and Story, Anaesth Intensive Care, 2005; Sporer, Drug Safety, 1995).

According to the Toxic Exposure Surveillance System (TESS), in 2002, there were 26,733 reported cases of exposure to selective serotonin-reuptake inhibitors (SSRIs) that caused serious toxic effects in 7,349 individuals and resulted in 93 fatalities (Isbister et al, J Toxicol Clin Toxicol 2004; Watson et al, Am J Emerg Med 2003).

The prevalence of the serotonin syndrome has relied on post-marketing surveillance reports, one of which identified an incidence of 0.4 per 1000 patient-months for patients who were taking the antidepressant nefazodone (Mackay et al, Br J Gen Pract 1999). The precise prevalence of the serotonin syndrome is difficult to assess; it is reported that approximately 85 percent of clinicians are unaware of the serotonin syndrome as a clinical diagnosis ((Mackay et al, Br J Gen Pract 1999). The serotonin syndrome occurs in approximately 14 to 16 percent of individuals who overdose on SSRIs (Isbister et al, J Toxicol Clin Toxicol 2004).

Clarkson et al (J Forensic Sci, 2004) reviewed a series of 66 deaths in Washington State between 1995-2000 in which tramadol was detected in the decedent's blood, in order to assess the role tramadol was determined to have played. Tramadol is an analgesic that exerts its effects through inhibition of reuptake of serotonin and norepinephrine, and through opioid agonism. Tramadol was consistently found together with other analgesic, muscle relaxant, and CNS depressant drugs. The investigators found that death was rarely attributable to tramadol alone. However, tramadol was a significant contributor to lethal intoxication when taken in excess with other drugs, via the potential interaction with serotonergic antidepressant medications (e.g., amitriptyline, nortriptyline, trazadone).

Serotonin syndrome can occur with the (i) initiation of therapy with a proserotonergic agent; (ii) the addition of a second proserotonergic agents; and (iii) intentional or accidental overdose with one or several proserotonergic agents. Proserotonergic agents are frequently used in patients with primary psychopathology (major depression, schizophrenia), in individuals who have chronic pain and those with chronic pain and comorbid depression or other affective disorders. Such populations are particularly predisposed to concomitant therapy with multiple proserotonergic drugs, other polypharmacy, drug and alcohol abuse and suicidal ideation. Consequently, patients receiving proserotonergic agents are at particular risk for accidental or intentional overdose with one or several prescribed or street drugs implicated in the serotonin syndrome.

Proserotonergic agents are also frequently used as primary therapy or in combination with conventional analgesics for the treatment of painful peripheral neuropathic pain (e.g., painful diabetic neuropathy, postherpetic neuralgia, etc) and central neuropathic pain (e.g, spinal cord injury pain, post-stroke pain, etc).

Over the past decade, there has been a growing appreciation of the value of extended release (also know as sustained release, controlled release and modified release) formulations in improving patient convenience and compliance for chronic conditions such as depression or chronic pain. Conventional (so called “immediate-release” or “short acting”) medications provide short-lived plasma levels, thereby requiring frequent dosing during the day (e.g., 4, 6 or 8 hours) to maintain therapeutic plasma levels of drug. In contrast, extended release formulations are designed to maintain effective plasma levels throughout a 12 or 24-hour dosing interval. Extended release formulations result in fewer interruptions in sleep, reduced dependence on caregivers, improved compliance, enhanced quality of life outcomes, and increased control over the management of their disease. In addition, such formulations can provide more constant plasma concentrations and clinical effects, less frequent peak to trough fluctuations and fewer side effects, compared with short acting drugs (Sloan and Babul, Expert Opinion on Drug Delivery 2006; Babul et al. Journal of Pain and Symptom Management 2004; 28:59-71; Matsumoto et al., Pain Medicine 2005; 6:357-66; Dhaliwal et al., Journal of Pain Symptom Management 1995; 10:612-23; Hays et al., Cancer 1994; 74:1808-16; Arkinstall et al., Pain 1995; 64:169-78; Hagen et al., Journal of Clinical Pharmacology 1995; 35:38-45; Peloso et al., Journal of Rheumatology 2000; 27:764-71).

However, such medications are not without drawbacks. Commercially available immediate-release formulations are designed to release a small amount of drug into the systemic circulation over several hours. New, extended release formulations are designed to gradually release their much larger drug load over a 12 or 24-hour period. Experience with extended release formulations of the pain reliever, oxycodone (OxyContin®) has shown that intentional crushing, tampering or extraction of the active ingredient from the formulation by addicts and recreational drug users destroys the controlled-release mechanism and results in a rapid surge of drug into the bloodstream, with the entire 12 or 24-hour drug supply released immediately with potential for toxic effects.

In the case of proserotonergic drugs, accidental or intentional crushing or extraction or overdose will result in a surge of high blood levels (serotonin excess). Studies have demonstrated that serotonin excess, leading to the serotonin syndrome, may be a result of a single proserotonergic drug or more frequently, from the combined effect of multiple proserotonergic drugs (e.g., the analgesic tramadol with an SSRI). If not properly diagnosed and treated, serotonin syndrome can lead to life-threatening complications and death.

The onset of symptoms of the serotonin syndrome is usually rapid, with clinical manifestations frequently occurring within minutes after a change in medication or self-poisoning (Mason et al, Medicine, 2000). More than half the patients with the serotonin syndrome present within six hours after initial use, misuse or abuse of medication, an overdose, or a change in dosing. (Mason et al, Medicine, 2000). Patients with mild symptoms may present with subacute or chronic symptoms, while those with severe intoxication may progress rapidly to death. It is believed that the serotonin syndrome does not resolve spontaneously as long as precipitating agents continue to be administered.

Management of the serotonin syndrome involves the removal of the precipitating drugs, supportive care, control of agitation, administration of 5-HT_{2A} antagonists and control of autonomic instability and any hyperthermia. Many cases of the serotonin syndrome typically resolve within 24 hours after the initiation of therapy and the discontinuation of serotonergic drugs, but symptoms may persist in patients taking drugs with long elimination half-lives, active metabolites, or a protracted duration of action. (Boyer and Shannon, NEJM, 2005; Jones and Story, Anaesth Intensive Care, 2005; Sporer, Drug Safety, 1995).

According to a recent state of the art review Boyer and Shannon, NEJM, March 2005), the serotonin syndrome can be avoided by “a combination of pharmacogenomic research, the education of physicians, modifications in prescribing practices, and the use of technological advances. The application of pharmacogenomic principles can potentially protect patients at risk for the syndrome before the administration of serotonergic agents. Once toxicity occurs, consultation with a medical toxicologist, a clinical pharmacology service, or a poison-control center can identify proserotonergic agents and drug interactions, assist clinicians in anticipating adverse effects, and provide valuable clinical decision-making experience. The avoidance of multidrug regimens is critical to the prevention of the serotonin syndrome. If multiple agents are required, however, computer-based ordering systems and the use of personal digital assistants can detect drug interactions and decrease reliance on memory in drug ordering. Post-marketing surveillance linked to physician education has been proposed to improve awareness of the serotonin syndrome.”

There is no prior art on pharmaceutical formulations that have a reduced risk of producing serotonin excess which is the cause of the serotonin syndrome.

### BRIEF SUMMARY OF THE INVENTION

The present invention is directed at pharmaceutical compositions and the use thereof for preventing or minimizing the risk of the serotonin syndrome. There are no methods in the literature directed at the development or use of pharmaceutical formulations that have a reduced risk of producing an excess of proserotonergic agents, which are the cause of the serotonin syndrome.

The present invention is directed at pharmaceutical dosage forms that prevent or reduce the intensity of the serotonin syndrome by reducing the amount of pros erotonergic agents accidentally or intentionally released into the systemic circulation.

Surprisingly, proserotonergic agents, including selective serotonin-reuptake inhibitors (SSRIs), e.g., citalopram, ecitalopram, fluoxetine, fluvoxamine, nefazodone, paroxetine, and sertaline; selective serotonin-norepinephrine reuptake inhibitors (SNRIs), e.g, bicifadine, venlafaxine, milnacipran, mirtazepine and nefazodone; tricyclic and non-tricyclic antidepressants, e.g., buspirone, clomipramine, trazadone; monoamine oxidase (MAO) inhibitors, e.g., clorgiline, isocarboxazid, moclobemide, phenelzine and selegiline; antiepileptics, e.g., valproate; analgesics, e.g., anileridine, dezocine, dihydrocodeine, hydrocodone, hydromorphone, fentanyl, levorphanol, meperidine, pentazocine, propiram, tramadol, other opioid analgesics; antiemetic agents, e.g., granisetron, metoclopramide and ondansetron; antimigraine drugs, e.g., sumatriptan; bariatric medications, e.g., sibutramine; antibiotics, e.g., linezolide (a MAOI) and ritonavir (via CYP-450 3A4 inhibition); antitussives, e.g. dextromethorphan; dietary supplements and herbal products, e.g., tryptophan, Hypericum perforatum (St. John's wort), Panax ginseng (ginseng); and lithium can be formulated and used with a reduced risk of the serotonin syndrome in the setting of accidental or intentional overdose and co-ingestion with other proserotonergic agents.

Both immediate release and extended release proserotonergic formulations can produce a serotonin surge when taken accidentally or intentionally in therapeutic, non-medical and overdose settings and when co-ingested with other proserotonergic agents.

Surprisingly, serotonin surge protector (SSP) formulations can reduce the incidence and severity of the serotonin syndrome.

A first aspect of the present invention is directed to a novel method for reducing the peak concentration of proserotonergic agent, said method comprising administering a proserotonergic agent and a suitable amount of SSP.

A second aspect of the present invention is directed to a novel method for reducing the area under the plasma concentration time curve (AUC) of proserotonergic agent, said method comprising administering a proserotonergic agent and a suitable amount of SSP.

A third aspect of the present invention is directed to a novel method for reducing the average plasma concentration time (Cave) of proserotonergic agent, said method comprising administering a proserotonergic agent and a suitable amount of SSP.

A fourth aspect of the present invention is directed to a novel method for reducing the incidence of the serotonin syndrome, said method comprising administering a proserotonergic agent and a suitable amount of SSP.

A fifth aspect of the present invention is directed to a novel method for reducing the intensity of the serotonin syndrome, said method comprising administering a proserotonergic agent and a suitable amount of SSP.

A sixth aspect of the present invention is directed to a novel method for reducing the intensity or frequency of one or more symptoms of the serotonin syndrome, including hyperthermia, tachycardia, shivering, diaphoresis, mydriasis, tremor, myoclonus, hyperreflexia, hypertension, hyperactive bowel sounds, agitation, hypervigilance, pressured speech, delirium, muscular rigidity, hypertonicity, metabolic acidosis, rhabdomyolysis, elevated levels of AST, ALT and creatinine, seizures, renal failure, and disseminated intravascular coagulopathy (DIC), said method comprising administering a proserotonergic agent and a suitable amount of SSP.

A seventh aspect of the present invention is directed to novel pharmaceutical compositions of matter for use in reducing the peak concentration of proserotonergic agent, said method comprising administering a proserotonergic agent and a suitable amount of SSP.

An eighth aspect of the present invention is directed to novel pharmaceutical compositions of matter for reducing the area under the plasma concentration time curve (AUC) of proserotonergic agent, said method comprising administering a proserotonergic agent and a suitable amount of SSP.

A ninth aspect of the present invention is directed to novel pharmaceutical compositions of matter for reducing the average plasma concentration time (Cave) of proserotonergic agent, said method comprising administering a proserotonergic agent and a suitable amount of SSP.

A tenth aspect of the present invention is directed to novel pharmaceutical compositions of matter for reducing the incidence of the serotonin syndrome, said method comprising administering a proserotonergic agent and a suitable amount of SSP.

An eleventh aspect of the present invention is directed to novel pharmaceutical compositions of matter for reducing the intensity of the serotonin syndrome, said method comprising administering a proserotonergic agent and a suitable amount of SSP.

An twelfth aspect of the present invention is directed to novel pharmaceutical compositions of matter for reducing the intensity or frequency of one or more symptoms of the serotonin syndrome, including hyperthermia, tachycardia, shivering, diaphoresis, mydriasis, tremor, myoclonus, hyperreflexia, hypertension, hyperactive bowel sounds, agitation, hypervigilance, pressured speech, delirium, muscular rigidity, hypertonicity, metabolic acidosis, rhabdomyolysis, elevated levels of AST, ALT and creatinine, seizures, renal failure, and disseminated intravascular coagulopathy (DIC), said method comprising administering a proserotonergic agent and a suitable amount of SSP.

### DETAILED DESCRIPTION OF THE INVENTION

The present invention relates to pharmaceutical compositions and the use thereof for preventing or minimizing the risk of the serotonin syndrome through the use of serotonin surge protectors (SSP).

SSP are pharmaceutical compositions which include one or more polymeric and/or nonpolymeric gel forming agents, viscosity increasing and/or high viscosity liquids, and optionally one or more excipients and inert carriers, that resist, deter or prevent crushing, shearing, grinding, chewing, dissolving, melting, needle aspiration, inhalation, insufflation or solvent extraction of the proserotonergic agent responsible for causing the serotonin syndrome through serotonin excess. SSP prevent or reduce the incidence and intensity of the serotonin syndrome when combined in the same formulation with one or more proserotonergic agents.

The present invention is related to pharmaceutical compositions comprising a proserotonergic agent alone or in combination with other therapeutic agents, one or more SSP, and optionally one or more excipients and inert carriers.

Compositions and methods of the present invention can form a viscous gel upon contact with a solvent such that the gel and proserotonergic agent cannot be easily drawn into a syringe, crushed to facilitate or enhance nasal delivery (snorting or nasal insufflation), inhalation or rapid oral delivery of a larger than planned delivery of the proserotonergic agent, such as to cause the serotonin syndrome.

In one embodiment of the invention, the SSP resists the release of all or substantially all of the proserotonergic contents of the unit dose. In another embodiment of the invention, the SSP resists the release of a portion of the proserotonergic contents of the unit dose. In yet another embodiment of the invention, the proserotonergic agent formulated with the SSP by a practitioner of the art resists the release the proserotonergic agent to a greater extent than when formulated without the SSP.

In some embodiments, the present invention is directed to oral dosage forms with an intended therapeutic effect of up to about 1 hour comprising (i) a proserotonergic agent and (ii) a serotonin surge protector.

In some embodiments, the present invention is directed to oral dosage forms with an intended therapeutic effect of up to about 2 hours comprising (i) a proserotonergic agent and (ii) a serotonin surge protector.

In some embodiments, the present invention is directed to oral dosage forms with an intended therapeutic effect of up to about 4 hours comprising (i) a proserotonergic agent and (ii) a serotonin surge protector.

In some embodiments, the present invention is directed to oral dosage forms with an intended therapeutic effect of up to about 6 hours comprising (i) a proserotonergic agent and (ii) a serotonin surge protector.

In some embodiments, the present invention is directed to oral dosage forms with an intended therapeutic effect of up to about 8 hours comprising (i) a proserotonergic agent and (ii) a serotonin surge protector.

In some embodiments, the present invention is directed to oral dosage forms with an intended therapeutic effect of up to about 12 hours comprising (i) a proserotonergic agent and (ii) a serotonin surge protector.

In some embodiments, the present invention is directed to oral dosage forms with an intended therapeutic effect of up to about 24 hours comprising (i) a proserotonergic agent and (ii) a serotonin surge protector.

In some preferred embodiments, the oral dosage form of the present invention is directed to an oral dosage form comprising (i) a proserotonergic agent and (ii) a serotonin surge protector, such that the ratio of the mean Cₘₐₓ of the proserotonergic agent following single dose oral administration of the dosage form after intentional or inadvertent tampering to the mean Cₘₐₓ of the proserotonergic agent after single dose oral administration of an intact dosage form is less than 10:1. In other embodiments of the invention, the mean Cₘₐₓ ratio using the aforementioned test method is at less than 7:1, 5:1, 3:1, 2:1 or 1.5:1.

In some preferred embodiments, the oral dosage form of the present invention is directed to an oral dosage form comprising (i) a proserotonergic agent and (ii) a serotonin surge protector, such that the ratio of the mean Tₘₐₓ of the proserotonergic agent following single dose oral administration of the dosage form after intentional or inadvertent tampering to the mean Tₘₐₓ of the proserotonergic agent after single dose oral administration of an intact dosage form is less than 10:1. In other embodiments of the invention, the mean Tₘₐₓ ratio using the aforementioned test method is at less than 7:1, 5:1, 3:1, 2:1 or 1.5:1.

In some preferred embodiments, the oral dosage form of the present invention is directed to an oral dosage form comprising (i) a proserotonergic agent and (ii) a serotonin surge protector, such that the ratio of the mean AUC₀₋₂ of the proserotonergic agent following single dose oral administration of the dosage form after intentional or inadvertent tampering to the mean AUC₀₋₂ of the proserotonergic agent after single dose oral administration of an intact dosage form is less than 10:1. In other embodiments of the invention, the mean AUC₀₋₂ ratio using the aforementioned test method is at less than 7:1, 5:1, 3:1, 2:1 or 1.5:1.

In some preferred embodiments, the oral dosage form of the present invention is directed to an oral dosage form comprising (i) a proserotonergic agent and (ii) a serotonin surge protector, wherein the amount of said proserotonergic agent released from the intact dosage form based on the dissolution at 1 hour of the dosage form in 900 mL of Simulated Gastric Fluid using a USP Type II (rotating paddle method) apparatus at 50 rpm at 37 degrees ° C. is 33% or less from the intact dosage form and 50% or less from the tampered dosage form. In other embodiments of the invention, the release rate using the aforementioned test method is 25% or less from the intact dosage form and 50% or less from the tampered dosage form, or 25% or less from the intact dosage form and 33% or less from the tampered dosage form or 20% or less from the intact dosage form and 33% or less from the tampered dosage form, or 15% or less from the intact dosage form and 25% or less from the tampered dosage form.

In one embodiment of the SSP, the therapeutic pharmaceutical composition can be formed into a unit dose including a proserotonergic agent and a gel forming polymer. In one embodiment, the polymer includes one or more of polyethylene oxide (e.g., having average molecular weight ranging form about 200,000 to about 5,000,000), polyvinyl alcohol (e.g., having a molecular weight of about 10,000 to 300,000) and hydroxypropyl methyl cellulose (e.g., having a molecular weight of about 10,000 to 1,700,000), and a carbomer (e.g., having a molecular weight ranging of about 600,000 to 4,000,000,000).

As described above, the present invention can include one or more gel forming agents. The total amount of gel forming agent is typically about 2 to about 80 percent, preferably 3 to 60 percent and more preferably 5 to 50 percent on a dry weight basis of the composition.

Suitable gel forming agents include compounds that, upon contact with a solvent (e.g., water), absorb the solvent and swell, thereby forming a viscous or semiviscous substance that significantly reduces and/or minimizes the amount of free solvent which can contain an amount of solubilzed drug. The gel can also reduce the overall amount of drug extractable with the solvent by entrapping the drug in a gel matrix. In one embodiment, typical gel forming agents include pharmaceutically acceptable polymers, typically hydrophilic polymers, such as hydrogels.

In some embodiments, the polymers exhibit a high degree of viscosity upon contact with a suitable solvent. The high viscosity can enhance the formation of highly viscous gels when attempts are made by to crush and dissolve the contents of a dosage form in an aqueous vehicle and inject it intravenously.

More specifically, in certain embodiments the polymeric material in the present invention provides viscosity to the dosage form when it is tampered. In such embodiments, when the composition is crushed and attempts are made to dissolve the dosage form in a solvent (e.g., water or saline), a viscous or semi-viscous gel is formed. The increase in the viscosity of the solution discourages injection of the gel by preventing the transfer of sufficient amounts of the solution to a syringe.

Suitable polymers include one or more pharmaceutically acceptable polymers selected from any pharmaceutical polymer that will undergo an increase in viscosity upon contact with a solvent. Preferred polymers include polyethylene oxide, polyvinyl alcohol, hydroxypropyl methyl cellulose and carbomers.

In some embodiments, the polymer includes polyethylene oxide. The polyethylene oxide can have an average molecular weight ranging from about 200,000 to about 5,000,000, more preferably from about 600,000 to about 5,000,000. In one embodiment, the polyethylene oxide includes a high molecular weight polyethylene oxide.

In one embodiment, the average particle size of the polyethylene oxide ranges from about 700 to about 2,000 microns. In another embodiment, the density of the polyethylene oxide can range from about 1.0 to about −1.35 g/mL. In another embodiment, the viscosity can range from about 8.00 to about 18,000 cps.

The polyethylene oxide used in a directly compressible formulation of the present invention is preferably a homopolymer having repeating oxyethylene groups, i.e., —(—O—CH₂.CH₂)ₙ—, where n can range from about 2,000 to about 180,000. Preferably, the polyethylene oxide is a commercially available and pharmaceutically acceptable homopolymer having moisture content of no greater than about 1% by weight. Examples of suitable, commercially available polyethylene oxide polymers include Polyox®, WSRN-1105 and/or WSR-coagulant.

In some embodiments, the polyethylene oxide powdered polymers can contribute to a consistent particle size in a directly compressible formulation and eliminate the problems of lack of content uniformity and possible segregation.

In one embodiment, the gel forming agent includes polyvinyl alcohol. The polyvinyl alcohol can have a molecular weight ranging from about 10,000 to about 300,000. The specific gravity of the polyvinyl alcohol can range from about 1.10 to about 1.30 and the viscosity from about 3 to about 70 cps. The polyvinyl alcohol used in the formulation is preferably a water-soluble synthetic polymer represented by —(—C₂H₄O—)ₙ—, where n can range from about 400 to about 6,000. Examples of suitable, commercially available polyvinyl alcohol polymers include PVA.

In one embodiment, the gel forming agent includes hydroxypropyl methyl cellulose (Hypromellose). The hydroxypropyl methyl cellulose can have a molecular weight ranging from about 10,000 to about 1,700,000, and typically from about 4000 to about 12,000, i.e., a low molecular weight hydroxypropyl methyl cellulose polymer. The specific gravity of the hydroxypropyl methyl cellulose can range from about 1.10 to about 1.35, with an average specific gravity of about 1.25 and a viscosity of about 3500 to 6000. The hydroxypropyl methylcellulose used in the formulation can be a water-soluble synthetic polymer. Examples of suitable, commercially available hydroxypropyl methylcellulose polymers include Methocel K100LV and Methocel K4M.

In one embodiment, the present invention includes carbomers. The carbomers can have a molecular weight ranging from 600,000 to about 4,000,000,000. The viscosity of the polymer can range from about 3000 to about 40,000 cps. Examples of suitable, commercially available carbomers include carbopol 934P NF, carbopol 974P NF and carbopol 971P NF.

Following the teachings set forth herein, other suitable gel forming agents can include one or more of the following polymers: ethylcellulose, cellulose acetate, cellulose acetate propionate, cellulose acetate butyrate, cellulose acetate phthalate and cellulose triacetate, cellulose ether, cellulose ester, cellulose ester ether, and cellulose, acrylic resins comprising copolymers synthesized from acrylic and methacrylic acid esters, the acrylic polymer may be selected from the group consisting of acrylic acid and methacrylic acid copolymers, methyl methacrylate copolymers, ethoxyetlryl methacrylates, cyanoetlryl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate copolymers.

Any of the above described polymers can be combined together or combined with other suitable polymers, and such combinations are within the scope of the present invention.

In some embodiments, SSP consists of hydrophobic polymers, hydrophilic polymers, gums, protein derived materials, waxes, shellac, oils and mixtures thereof.

In one embodiment, the SSP can prevent less than or equal to about 95%, 94%, 70%, 60%, 54%, 50%, 45%, 40%, 36%, 32%, 30%, 27%, 20%, 10%, 9%, 6%, 5% or 2% of the total amount of drug in a dosage form from being recovered from a solvent in contact with a dosage form of the present invention.

The above described gel forming agents can be further optimized as necessary or desired in terms of viscosity, molecular weight, etc.

The present invention can also optionally include other ingredients to enhance dosage form manufacture from a pharmaceutical composition of the present invention and/or alter the release profile of a dosage forming including a pharmaceutical composition of the present invention.

Some embodiments of the present invention include one or more pharmaceutically acceptable fillers/diluents. In one embodiment, Avicel PH (Microcrystalline cellulose) is a filler used in the formulation. The Avicel PH can have an average particle size ranging from 20 to about 200 μm, preferably about 100 μm. The density ranges from 1.5 to 1.7 g/cm³. The Avicel PH should have molecular weight of about 36,000. Avicel PH effectiveness is optimal when it is present in an amount of from about 5 to 75 percent, by weight on a solid basis, of the formulation. Typical fillers can be present in amounts from 5 to 75 percent by weight on a dry weight basis. Other ingredients can include sugars and/or polyols.

Other ingredients can also include dibasic calcium phosphate having a particle size of about 60 to about 450 microns and a density of about 0.5 to about 1.5 g/ml, as well as calcium sulfate having a particle size of about 1 to about 200 microns and a density of about 0.6 to about 1.3 g/ml and mixtures thereof. Further, lactose having a particle size of about 20 to about 400 microns and a density of about 0.3 to about 0.9 g/ml can also be included.

In some embodiments of the invention, the fillers which can be present at about 5 to 85 percent by weight on a dry weight basis, also function as binders in that they not only impart cohesive properties to the material within the formulation, but can also increase the bulk weight of a directly compressible formulation (as described below) to achieve an acceptable formulation weight for direct compression. In some embodiments, additional fillers need not provide the same level of cohesive properties as the binders selected, but can be capable of contributing to formulation homogeneity and resist segregation from the formulation once blended. Further, preferred fillers do not have a detrimental effect on the flowability of the composition or dissolution profile of the formed tablets.

In one embodiment, the present invention can include one or more pharmaceutically acceptable disintegrants. Such disintegrants are known to those skilled in the art. In the present invention, disintegrants can include, but are not limited to, sodium starch glycolate having a particle size of about 100 microns and a density of about 0.75 g/mL, starch (e.g., Starch 21) having a particle size of about 2 to about 32 microns and a density of about 0.46 g/ml, Crospovidone® having a particle size of about 400 microns and a density of about 1.2 g/ml, and croscarmellose sodium (Ac-Di-Sol) having a particle size of about 37 to about 73.7 microns and a density of about 0.53 g/ml. The disintegrant selected should contribute to the compressibility, flowability and homogeneity of the formulation. Further the disintegrant can minimize segregation and provide an immediate release profile to the formulation. In some embodiments, the disintegrant(s) are present in an amount from about 2 to about 35 percent by weight on a solid basis of the directly compressible formulation.

In one embodiment, the present invention can include one or more pharmaceutically acceptable glidants, including but not limited to colloidal silicon dioxide. In one embodiment, colloidal silicon dioxide (Cab-O-Sil®) having a density of about 0.023 to about 0.040 g/ml can be used to improve the flow characteristics of the formulation. Such glidants can be provided in an amount of from about 0.1 to about 3 percent by weight of the formulation on a solid basis. It will be understood, based on this invention, however, that while colloidal silicon dioxide is one particular glidant, other glidants having similar properties which are known or to be developed could be used provided they are compatible with other excipients and the active ingredient in the formulation and which do not significantly affect the flowability, homogeneity and compressibility of the formulation.

In one embodiment, the present invention can include one or more pharmaceutically acceptable lubricants, including but not limited to magnesium stearate. In one embodiment, the magnesium stearate has a particle size of about 450 to about 550 microns and a density of about 1.0 to about 1.8 g/ml. In one embodiment, magnesium stearate can contribute to reducing friction between a die wall and a pharmaceutical composition of the present invention during compression and can ease the ejection of the tablets, thereby facilitating processing. In some embodiments, the lubricant resists adhesion to punches and dies and/or aid in the flow of the powder in a hopper and/or into a die. In an embodiment of the present invention, magnesium stearate having a particle size of from about 5 to about 50 microns and a density of from about 0.1 to about 1.1 g/ml is used in a pharmaceutical composition. In certain embodiments, a lubricant should make up from about 0.1 to about 2 percent by weight of the formulation on a solids basis. Suitable lubricants are stable and do not polymerize within the formulation once combined. Other lubricants known in the art or to be developed which exhibit acceptable or comparable properties include stearic acid, hydrogenated oils, sodium stearyl fumarate, polyethylene glycols, and Lubritab®.

In certain embodiments, the most important criteria for selection of the excipients are that the excipients should achieve good content uniformity and release the active ingredient as desired. The excipients, by having excellent binding properties, and homogeneity, as well as good compressibility, cohesiveness and flowability in blended form, minimize segregation of powders in the hopper during direct compression.

Compositions and methods of the SSP can also be selected from a group of viscosity-increasing agents selected from the group consisting of microcrystalline cellulose with 11 wt. % carboxymethylcellulose sodium (e.g., Avicel® RC 591), carboxymethylcellulose sodium (e.g., Blanose®, CMC-Na C3001P®, Frimulsion BLC-5®, Tylose C300 P®), polyacrylic acid (e.g., Carbopol® 980 NF, Carbopol® 981), locust bean flour (e.g., Cesagum® LA-200, Cesagum® LID/150, Cesagum® LN-1), pectins, preferably from citrus fruits or apples (e.g., Cesapectin® HM Medium Rapid Set), waxy maize starch (e.g., C*Gel 04201®), sodium alginate (e.g., Frimulsion ALG (E401)®), guar flour (e.g., Frimulsion BM®, Polygum 2611-75®), iota-carrageenan (e.g., Frimulsion D021®), karaya gum, gellan gum (e.g., Kelcogel F®, Kelcogel LT100®), galactomannan (e.g., Meyprogat 150®), tara stone flour (e.g., Polygum 4311®), propylene glycol alginate (e.g., ProtanalEster SD-LB®), sodium hyaluronate, tragacanth, tara gum (e.g., Vidogum SP 200®), fermented polysaccharide welan gum (K1A96), xanthans such as xanthan gum (e.g., Xantural 180®). The names stated in brackets are the trade names by which the materials are known commercially. In general, a quantity of 0.1 to 90% w/w, preferably of 1 to 70% w/w, particularly preferably of 5 to 50% w/w of the viscosity-increasing agent, relative to the total formulation, is sufficient in order to meet the requirements of SSP.

Surprisingly, in one embodiment, due to the inventive selection of the SSP, it is possible to combine the proserotonergic agents and the viscosity-increasing agents in the dosage form according to the invention without spatial separation from one another.

In another embodiment, the viscosity-increasing agents and the proserotonergic agents are contained in the dosage form in a mutually spatially separated arrangement.

In yet another embodiment of the present invention, the orally administrable dosage form according to the invention assumes multiparticulate form containing in each case the complete mixture of active ingredient and viscosity-increasing agent, preferably in the form of microtablets, microcapsules, micropellets, granules, spheroids, beads or pellets, preferably packaged in capsules or press-molded into tablets The multiparticulate forms preferably have a size in the range from 0.1 to 3 mm, particularly preferably in the range from 0.5 to 2 mm.

Compositions and methods of the present SSP invention can also be selected from a group of viscosity-increasing, gel-forming and solvent extraction resistant agents selected from the group consisting of hydrogenated Type I or Type II vegetable oils, polyoxyethylene stearates and distearates, glycerol monostearate (e.g., Cithrol® GMS) and poorly water soluble, high melting point (mp=40 to 100° C.) waxes.

Hydrogenated vegetable oils of the present invention may include hydrogenated cottonseed oil (e.g., Akofine®; Lubritab®; Sterotex® NF), hydrogenated palm oil (Dynasan® P60; Softisan® 154), hydrogenated soybean oil (Hydrocote®; Lipovol HS-K®; Sterotex® HM) and hydrogenated palm kernel oil (e.g., Hydrokote® 112).

Polyoxyethylene stearates and distearates of the present invention include Polyoxyl 2, 4, 6, 8, 12, 20, 30, 40, 50, 100 and 150 stearates (e.g., Hodag® DGS; PEG-2 stearate; Acconon® 200-MS; Hodag® 20-S; PEG-4 stearate; Cerasynt® 616; Kessco® PEG 300 Monostearate; Acconon® 400-MS; Cerasynt® 660; Cithrol® 4MS; Hodag® 60-S; Kessco® PEG 600 Monostearate; Cerasynt® 840; Hodag 100-S; Myrj® 51; PEG-30 stearate; polyoxyethylene (30) stearate; Crodet® S40; E431; Emerest® 2672; Atlas G-2153; Crodet® S50) and polyoxyl 4, 8, 12, 32 and 150 distearates (e.g, Lipo-PEG® 100-S; Myrj® 59; Hodag® 600-S; Ritox® 59; Hodag® 22-S; PEG-4 distearate; Hodag® 42-S; Kessco® PEG 400 DS; Hodag® 62-S; Kessco® PEG 600 Distearate; Hodag® 154-S; Kessco® PEG 1540 Distearate; Lipo-PEG® 6000-DS; Protamate® 6000-DS).

In one embodiment of the present invention, the proserotonergic agent is combined with beeswax, hydroxypropyl methyl cellulose (e.g, HPMC K15M), silicon dioxide (alone or in combination with Al₂O₃; e.g, Aerosil®, Aerosil® 200, Aerosil® COK84).

In one embodiment of the present invention, the proserotonergic agent is combined with hydrogenated cottonseed oil (e.g., Sterotex® NF), hydroxypropyl methyl cellulose (e.g, HPMC K15M), coconut oil and silicon dioxide (alone or in combination with Al₂O₃; e.g, Aerosil, Aerosil 200, Aerosil COK84).

In another embodiment of the present invention, the proserotonergic agent is combined with glycerol monostearate (e.g., Cithrol® GMS), hydroxypropyl methyl cellulose (e.g, HPMC K100M) and silicon dioxide (alone or in combination with Al₂O₃; e.g, Aerosil, Aerosil 200, Aerosil COK84).

In yet another embodiment of the present invention, the proserotonergic agent is combined with hydrogenated palm kernel oil (e.g., Hydrokote® 112), hydroxypropyl methyl cellulose (e.g, HPMC K15M) and silicon dioxide (alone or in combination with Al₂O₃; e.g, Aerosil, Aerosil 200, Aerosil COK84).

In one embodiment of the present invention, release rate modifiers, including hydroxypropyl methyl cellulose (e.g, HPMC K15M) may incorporated. Release rate modifiers can also have additional useful properties that optimize the formulation. For example HPMC is soluble in cool/cold water and becomes insoluble over approximately 40° C. This resists the generation of injectable solutions, interferes with ‘snorting’ or ‘dose dumping’ (due to the viscous solutions produced) and resists extraction at elevated temperature. A range of HPMCs of differing molecular weights and viscosities may be used with the present invention.

A variety of agents may incorporated into the invention as thixotropes (e.g., fumed silicon dioxides, Aerosil, Aerosil COK84, Aerosil 200, etc.). Thixotropes enhance the pharmaceutical formulations of the invention by increasing the viscosity of solutions during attempted extraction, complementing the action of HPMCs. They may also provide a tamper resistance by helping to retain the structure of dosage units that have been heated to temperatures greater than the melting point of the base excipient (Aerosils are unaffected by heat).

The dosage form according to the invention may preferably also comprise one or more proserotonergic agents, blended with the viscosity-increasing, gel-forming, high melting point waxes and solvent extraction resistant agents, at least in part in delayed-release form, wherein delayed release may be achieved with the assistance of conventional materials and processes known to the person skilled in the art, for example by embedding the active ingredient in a delayed-release matrix or by applying one or more delayed-release coatings.

Delayed release of the active ingredient may preferably also be achieved by purposeful selection of one or more of the above-stated viscosity-increasing agents in suitable quantities as the matrix material. The person skilled in the art may determine the agents and the quantity thereof suitable for the particular desired release by simple preliminary testing, wherein it must, of course, be ensured that, as described above, gel formation occurs when the attempt is made to abuse the resultant dosage form.

If the dosage form according to the invention is intended for oral administration, it may also comprise a coating which is resistant to gastric juices and dissolves as a function of the pH value of the release environment.

By means of this coating, it is possible to ensure that, when correctly administered, the dosage form according to the invention passes through the stomach undissolved and the active ingredient is only released in the intestines.

In another embodiment of the present invention, the formulation may comprise one or more proserotonergic agents blended with one or more high viscosity liquids. High viscosity liquids refers to non-polymeric, non-water soluble liquids with a viscosity of at least 5,000 cP at 37° C. that do not crystallize neat under ambient or physiological conditions. High viscosity liquids may be carbohydrate-based, and may include one or more cyclic carbohydrates chemically combined with one or more carboxylic acids, such as sucrose acetate isobutyrate. High viscosity liquids also include nonpolymeric esters or mixed esters of one or more carboxylic acids, having a viscosity of at least 5,000 cP at 37° C., that do not crystallize neat under ambient or physiological conditions, wherein when the ester contains an alcohol moiety (e.g., glycerol). The ester may, for example comprise from about 2 to about 20 hydroxy acid moieties.

The present invention may employ any high viscosity liquid, viscosity-enhancing compounds, gel-forming and solvent extraction resistant agents, not limited by any specifically described compounds.

In one embodiment of the invention, the formulation is ingested orally as a tablet or capsule, preferably as a capsule. In another embodiment of the invention, the formulation is administered bucally. In yet another embodiment of the invention, the formulation is administered sublingually.

In one embodiment of the invention, the dosage form includes a capsule within a capsule, each capsule containing a different drug or the same drug intended for a different purpose. In some embodiments, the outer capsule may be an enteric coated capsule or a capsule containing an immediate release formulation to provide rapid plasma concentrations or a rapid onset of effect or a loading dose and the inner capsule contains an extended release formulation. Up to 3 capsules within a capsule are contemplated as part of the invention in some embodiments. In one embodiment of the invention, the dosage form involves a tablet within a capsule, wherein the proserotonergic drug is either in the tablet and/or in one of the capsules.

“Drug,” “pharmacological agent,” “pharmaceutical agent,” “active agent,” and “agent” are used interchangeably and are intended to have their broadest interpretation as to any therapeutically active substance which is delivered to a living organism to produce a desired, usually beneficial effect. In general, this includes therapeutic agents in all of the major therapeutic areas, also including proteins, peptides, oligonucleotides, and carbohydrates as well as inorganic ions, such as calcium ion, lanthanum ion, potassium ion, magnesium ion, phosphate ion, and chloride ion.

“Pharmaceutically or therapeutically acceptable excipient or carrier” refers to a substance which does not interfere with the effectiveness or the biological activity of the active ingredients and which is not toxic to the hosts, which may be either humans or animals, to which it is administered. In some embodiments of the present invention, pharmaceutically or therapeutically acceptable excipients or carriers may play a role imparting or optimizing the SSP characteristics to the pharmaceutical composition.

“Therapeutically effective amount” refers to the amount of an active agent sufficient to induce a desired biological result. That result may be alleviation of the signs, symptoms, or causes of a disease, or any other desired alteration of a biological system.

The phrase “therapeutically-effective” is intended to qualify the amount of each agent which will achieve the goal of improvement in disease severity and the frequency of incidence over treatment of each agent by itself, while avoiding adverse side effects typically associated with alternative therapies.

The term “effective amount” means the quantity of a compound according to the invention necessary to prevent, to cure, or at least partially arrest a symptom for which the proserotonergic agent has been prescribed to a subject. A subject is any animal, preferably any mammal, more preferably a human.

As used herein the terms: (i) “AUC₀₋₂” means area under the drug concentration-time curve from time zero to two hours post-dose; (ii) “Cₘₐₓ” means the maximum observed drug concentration and (iii) “tₘₐₓ” or “Tₘₐₓ” means the time of the observed maximum drug concentration.

In certain embodiments, any one or all of the in-vivo pharmacokinetic parameters (e.g., AUC₀₋₂, Cₘₐₓ, Tₘₐₓ) are achieved after administration of the dosage form to a single human patient (i.e., an individual patient or subject).

In certain embodiments, any one or all of the in-vivo pharmacokinetic parameters (e.g., AUC₀₋₂, Cₘₐₓ, Tₘₐₓ) are achieved after administration of the dosage form to a population of patients; said population of patients consisting of two or more patients or subjects.

In certain embodiments, any one or all of the in-vivo pharmacokinetic parameters (e.g., AUC₀₋₂, Cₘₐₓ, Tₘₐₓ) are achieved after administration of the dosage form to a single human patient or subject or a population of patients or subjects in the fasted state

In certain embodiments, any one or all of the in-vivo pharmacokinetic parameters (e.g., AUC₀₋₂, Cₘₐₓ, Tₘₐₓ) are achieved after administration of the dosage form to a single human patient or subject or a population of patients or subjects in the fed state.

The term “opioid agonist” also referred to as “opioid receptor agonist” means a molecule that causes a specific physiologic, pathophysiologic or pharmacologic effect after binding to an opioid receptor, such actions a consequence of their agonist or agonistic effects. Opioid agonists are known or can be readily determined by individuals who skilled in the art.

The term “opioid receptor” includes mu (μ), delta (δ) and kappa (κ) opioid receptors, their subtypes and splice variants such as μ₁, μ₂, δ₁, δ₂, κ₁, κ₂ and κ₃, etc.

As used herein, the term “serotonin surge protector”, “SSP” or “SSP's” means pharmaceutical compositions that resist, deter or prevent crushing, shearing, grinding, chewing, dissolving, melting, needle aspiration, inhalation, insufflation or solvent extraction of the proserotonergic agent contained therein which is responsible for causing the serotonin syndrome through serotonin excess, thereby preventing or reducing the incidence and intensity of the serotonin syndrome when the SSP is combined in the same formulation with one or more proserotonergic agents. Preferred SSP's are selected from a group consisting of polymeric and/or nonpolymeric gel forming agents; viscosity enhancing agents, high viscosity liquids and high melting point waxes, hydrogenated Type I or Type II vegetable oils, polyoxyethylene stearates and distearates, glycerol monostearate, and non-polymeric, non-water soluble liquids, carbohydrate-based substances or poorly water soluble, high melting point (mp=40 to 100° C.) waxes and mixtures thereof. In some embodiments, SSP's include polyethylene oxides, polyvinyl alcohol, hydroxypropyl methyl cellulose, carbomers, ethylcellulose, cellulose acetate, cellulose acetate propionate, cellulose acetate butyrate, cellulose acetate phthalate and cellulose triacetate, cellulose ether, cellulose ester, cellulose ester ether, and cellulose, acrylic resins comprising copolymers synthesized from acrylic and methacrylic acid esters, the acrylic polymer may be selected from the group consisting of acrylic acid and methacrylic acid copolymers, methyl methacrylate copolymers, ethoxyetlryl methacrylates, cyanoetlryl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), glycidyl methacrylate copolymers, microcrystalline cellulose with carboxymethylcellulose sodium, carboxymethylcellulose sodium, polyacrylic acid, locust bean flour, pectins, waxy corn starch, sodium alginate, guar flour, iota-carrageenan, karaya gum, gellan gum, galactomannan, tara stone flour, propylene glycol alginate, sodium hyaluronate, tragacanth, tara gum, fermented polysaccharide welan gum, xanthans, silicon dioxide, fumed silicon dioxide, coconut oil, hydrogenated palm kernel oil, hydrogenated cottonseed oil, hydrogenated palm kernel oil, hydrogenated palm oil, hydrogenated soybean oil and beeswax, and mixtures thereof. In some embodiments, SSP's include hydrophobic polymers, hydrophilic polymers, gums, protein derived materials, waxes, shellac, oils and mixtures thereof.

As used herein and without being bound by theory, the term “proserotonergic agent(s)” means drugs that directly or indirectly enhance the effects of serotonin, usually through reuptake inhibition, direct or indirect agonism, enhancement of effects of serotonergic drugs or other known or unknown mechanism, such that they have the potential to produce the serotonin syndrome, said serotonin syndrome characterized by one or more adverse signs and symptoms, including hyperthermia, tachycardia, shivering, diaphoresis, mydriasis, tremor, myoclonus, hyperreflexia, hypertension, hyperactive bowel sounds, agitation, hypervigilance, pressured speech, delirium, muscular rigidity, hypertonicity, metabolic acidosis, rhabdomyolysis, elevated levels of AST, ALT and creatinine, seizures; renal failure, and disseminated intravascular coagulopathy (DIC). For the purposes of the invention, proserotonergic drugs include drugs selected from a group consisting of selective serotonin-reuptake inhibitors (SSRIs), selective serotonin-norepinephrine reuptake inhibitors (SNRIs), serotonin reuptake inhibitors, norepinephrine reuptake inhibitors, tricyclic, tetracyclic and non-tricyclic antidepressants, monoamine oxidase (MAO) inhibitors, antiepileptics, opioid analgesics, tramadol, antiemetics, bariatric medications, sibutramine, antibiotics, antimigraine drugs, antivirals, and cough suppressants, and mixtures thereof given in the form of an acid, base or, optionally, in the form of a pharmaceutically acceptable salt, prodrug, ester, analog, derivative, solvate, complex, polymorph, hydrate, racemate or an individual diastereoisomers or enantiomeric isomers thereof or mixture thereof. In some embodiments, the proserotonergic agent includes citalopram, fluoxetine, fluvoxamine, paroxetine, sertaline, venlafaxine, milnacipran, buspirone, clomipramine, nefazodone, trazadone, clorgiline, isocarboxazid, moclobemide, phenelzine, selegiline, valproate, fentanyl, levorphanol, meperidine, pentazocine, tramadol, granisetron, metoclopramide, ondansetron, sumatriptan, sibutramine, linezolide, ritonavir, dextromethorphan, dextrorphan, tryptophan, hypericum perforatum (St. John's wort), panax ginseng (ginseng) and lithium.

Without being bound by theory, proserotonergic agents include selective serotonin-reuptake inhibitors (SSRIs), e.g., citalopram, ecitalopram, fluoxetine, fluvoxamine, nefazodone, paroxetine, and sertaline; selective serotonin-norepinephrine reuptake inhibitors (SNRIs), e.g, bicifadine, venlafaxine, milnacipran, mirtazepine and nefazodone; tricyclic and non-tricyclic antidepressants, e.g., buspirone, clomipramine, trazadone; monoamine oxidase (MAO) inhibitors, e.g., clorgiline, isocarboxazid, moclobemide, phenelzine and selegiline; antiepileptics, e.g., valproate; analgesics, e.g., fentanyl, levorphanol, meperidine, pentazocine, tramadol, other opioid analgesics (see below); antiemetic agents, e.g., granisetron, metoclopramide and ondansetron; antimigraine drugs, e.g., sumatriptan; bariatric medications, e.g., sibutramine; antibiotics, e.g., linezolide (a MAOI) and ritonavir (via CYP-450 3A4 inhibition); antitussives, e.g. dextromethorphan; dietary supplements and herbal products, e.g., tryptophan, Hypericum perforatum (St. John's wort), Panax ginseng (ginseng); lithium; and drugs that are serotonin receptor agonists.

Opioid analgesics include alfentanil, allylprodine, alphaprodine, anileridine, apomorphine, apocodeine, benzylmorphine, bezitramide, buprenorphine, butorphanol, carfentanil, clonitazene, codeine, cyclazocine, cyclorphen, cyprenorphine, desmethyltramadol, desomorphine, dextromoramide, dezocine, diampromide, dihydrocodeine, dihydromorphine, dimenoxadol, dimepheptanol, dimethylthiambutene, dioxyaphetyl butyrate, dipipanone, eptazocine, ethoheptazine, ethylmethylthiambutene, ethylmorphine, etonitazene, fentanyl, heroin, hydrocodone, hydroxymethylmorphinan, hydromorphone, hydroxypethidine, isomethadone, ketobemidone, levallorphan, levorphanol, levomethadone, levophenacylmorphan, lofentanil, meperidine, meptazinol, metazocine, methadone, methylmorphine, metopon, morphine, myrophine, nalbuphine, narceine, nicomorphine, norlevorphanol, normethadone, nalorphine, normorphine, norpipanone, ohmefentanyl, opium, oxycodone, oxymorphone, papavereturn, pentazocine, phenadoxone, phenomorphan, phenazocine, phenoperidine, pholcodine, piminodine, piritramide, propheptazine, promedol, profadol, properidine, propiram, propoxyphene, remifentanil, sufentanil, tapentadol, tramadol, tramadol metabolites, tilidine, naltrexone, naloxone, nalmefene, methylnaltrexone, naloxone methiodide, nalorphine, naloxonazine, nalide, nalmexone, nalbuphine, nalorphine dinicotinate, naltrindole (NTI), naltrindole isothiocyanate, (NTH), naltriben (NTB), nor-binaltorphimine (nor-BNI), racemorphan, beta-funaltrexamine (b-FNA), BNTX, cyprodime, ICI-174,864, LY117413, MR2266, etorphine, DAMGO, CTOP, diprenorphine, naloxone benzoylhydrazone, bremazocine, ethylketocyclazocine, U50,488, U69, 593, spiradoline, DPDPE, [D-Ala2,Glu4] deltorphin, DSLET, Met-enkephalin, Leu-enkephalin, (3-endorphin, dynorphin A, dynorphin B, a-neoendorphin, or an opioid having the same pentacyclic nucleus as nalmefene, naltrexone, buprenorphine, levorphanol, meptazinol, pentazocine or dezocine. Opioids include the unsalified drug or the pharmaceutically acceptable salts, esters, analogs, derivatives, solvates, complexes, polymorphs, hydrates, as racemates or an individual diastereoisomers or enantiomeric isomers thereof or mixture thereof.

Proserotonergic agents include the drugs, their pharmaceutically acceptable salts, esters, analogs, derivatives, solvates, complexes, polymorphs, hydrates, as a racemates or an individual diastereoisomers or enantiomeric isomers thereof or mixture thereof.

All modes of administration and co-administration are contemplated in the present invention, including oral, subcutaneous, direct intravenous, slow intravenous infusion, continuous intravenous infusion, intravenous or epidural patient controlled analgesia (PCA and PCEA), intramuscular, intrathecal, epidural, intracisternal, intramuscular, intraperitoneal, transdermal, topical, transmucosal, buccal, sublingual, transmucosal, inhalation, intranasal, epidural, intra-articular, intranasal, rectal or ocular routes.

The term “pharmaceutically acceptable salt” as used herein refers to a salt which is toxicologically safe for human and animal administration. Nonlimiting examples of salts include hydrochlorides, hydrobromides, hydroiodides, sulfates, bisulfates, nitrates, citrates, tartrates, bitartrates, phosphates, malates, maleates, napsylates, fumarates, succinates, acetates, terephthalates, pamoates and pectinates. Preferably, the pharmaceutically acceptable salt of levorphanol is a tartrate. Preferably, the pharmaceutically acceptable salt of morphine is a hydrochloride, a sulfate or a tartrate.

The present invention anticipates the use of more than one proserotonergic agent, given in the same formulation of in a different formulation, for use to treat, prevent or ameliorate the same disease or a different disease.

It is contemplated that the present invention may be used alone or in combination with other drugs to provide additive, complementary, or synergistic therapeutic effects, including other NSAIDs, COX-2 selective inhibitors, acetaminophen, tramadol, local anesthetics, beta adrenergic agonists, alpha-2 agonists, selective prostanoid receptor antagonists, cannabinoid receptor agonists, NMDA receptor antagonists, neuronal nicotinic receptor agonists, calcium channel antagonists, sodium channel blockers, superoxide dismutase mimetics, p38 MAP kinase inhibitors, TRPV1 agonists, antiepileptics, and any other drugs that can be shown by a person proficient in the art to prevent or treat pain. The drug being used in combination therapy with the present invention can be administered by any route, including parenterally, orally, topically, transdermally, sublingually, and the like.

In one embodiment of the invention, the proserotonergic drug tramadol is given alone as an immediate release formulation. In another embodiment, tramadol is given as an extended release formulation. In another embodiment, extended release tramadol is administered with fluoxetine. In another embodiment, levorphanol is administered alone as an extended release formulation. In another embodiment, extended release levorphanol is administered with venlafaxine.

The term “controlled release” as used herein is intended to distinguish it from immediate release dosage forms. Controlled release means a formulation or composition intended for any route of administration, including oral, buccal, rectal, transdermal, epidural, intramuscular, subcutaneous, inhaled and the like, which is prepared in such a manner as to allow for delayed, gradual, modulated and for prolonged release of the μ-opioid receptor agonist and/or levorphanol. As used herein, controlled release is interchangeable with “extended release”, “sustained release”, “pulsatile release”, “modified release”, “depot” and the like.

To further evaluate this invention, tramadol was selected as a prototype drug. Tramadol serves as an excellent prototype drug as it: 1) has been implicated when used alone in the serotonin syndrome; 2) has been implicated in the serotonin syndrome when used in combination with other drugs; 3) has significant proserotonergic and opioid effects, both of which have been implicated in the serotonin syndrome; 4) is water soluble and therefore prone to easy extraction and gastrointestinal absorption; 4) is available in both immediate release and extended release formulations, which if tampered with may dump an entire days contents into the systemic circulation, thereby increasing the incidence and severity of the serotonin syndrome.

Tramadol is a synthetic, centrally acting analgesic which exerts its analgesic effects by inhibiting reuptake of norepinephrine and serotonin and by activation of μ-opioid receptors. Tramadol binds to the μ-opioid receptor, although its principal active (M1) metabolite, mono-O-demethyl-tramadol is up to 6 times more potent in producing analgesia and 200 times more potent in μ-opioid binding (Ultram Package Insert). During its intentional or inadvertent non-medical use, tramadol, especially the extended release tramadol is likely to be crushed. Since tramadol produces dose dependent seizures and dose dependent serotonin syndrome, there is the potential for a compounded risk.

The occurrence of serotonin syndrome has been well documented with tramadol given alone, with serious and potentially fatal consequences (Clarkson et al, 2004; Garrett, 2004; Kitson and Carr, 2005). The sudden exposure of patients to large concentrations of tramadol from crushed solid dosage forms of Tramadol ER, especially in the face of ubiquitous use of SSRI's and SNRI's in chronic pain may have important medical consequences. (Clarkson et al, 2004; Gonzalez-Pinto et al, 2001; Houlihan, 2004; Egberts et al, 1997; Kesavan and Sobala, 1999; Lange-Asschenfeldt, 2002; Mahlberg et al, 2004; Mittino et al, 2004).

### EXAMPLES

Materials in the series of experiments below included the following: Aerosil 200, Lot 1412033, ex Degussa Huls, Aerosil COK84, Lot 2258, ex Degussa, Huls, Beeswax, Lot A018035701, ex Acros Organics, Cetyl alcohol (1-hexadecanol), Lot A019258301, ex Acros Organics, Cithrol GMS 0400, Lot 6483-0103, ex Croda, Fractionated coconut oil, Lot 165544, ex A E Connock Gelucire 44/14, Lot 22009, ex Gattefosse, Gelucire 50/02, Lot 19255, ex Gattefosse, Gelucire 50/13, Lot 20529, ex Gattefosse, Hydrokote 112 Lot 048M3, ex Abitech Corp, Hydrokote APS, Lot 340J1, ex Abitech Corp, Hydrokote M, Lot 126J2, ex Abitech Corp, Methocel AM4, Lot Q101012N01, ex Colorcon, Methocel K100M, Lot QA15012N01, ex Colorcon, Methocel K15M, Lot QK02012N11, ex Colorcon, Paraffin wax, Lot P/0680/90, ex Fisher Scientific, PEG 400, Lot 310354, ex NOF Corp, Pluriol E6005 (PEG 6000), Lot 97193, ex BASF, Pharmacoat 606 (hypromellose USP), Lot 308522, ex Shin-Etsu Chemical Co Ltd., Poloxamer 124 (Pluronic 144), Lot WPWV-645B, ex BASF., Poloxamer 188 (Lutrol F68), Lot 0306043523, ex BASF, Propylene glycol, Lot 09521110, ex Aldrich, Propranolol HCl, Lot 044K1219, ex Sigma, Shellac, Lot 4010 2465 2056, ex Syntapharm, Size 1 clear/clear gelatin capsules, Lot C14893, ex Capsugel, Starch 1500, Lot IN 500578, ex Colorcon, Sterotex N F, Lot 324M2, ex Abitech Corp., Tramadol HCl, Lot 3TRMDN0D105 & 3TRMDN0E056, ex Chemagis Ltd, Zein (Paroxite), Lot 5041C, ex Variati & Co.

Equipment in the series of experiments below included the following: Caleva 9ST dissolution apparatus with ERWEKA P thermostatically controlled water heater, Copley ZT54 disintegration apparatus, Haake DC5 water bath, Heidolph bench mixer, HiBar bench filling machine, Qualiseal bench banding machine, Silverson SL2 bench high shear mixer, Thermo Electron Vision uv/visible spectrometry data acquisition program with Vision Security, Unicam UV2-400 spectrophotometer, Watson Marlow 205U peristaltic pump 650μ nominal s/s Laboratory test sieve, 600μ s/s certified Laboratory test sieve from Endecotts Ltd, London, Whatman 25 mm 45μ filters used in combination with a 5 ml Luer lock syringe.

### Example 1

### Binary Mix Compatibility Trials

Binary mixes were prepared of tramadol HCL in potential excipients (in some instances a third material, fractionated coconut oil was used to bring two non melting materials into intimate contact). The mixes were stored in sealed amber glass bottles under conditions of 40° C./75% RH for four weeks then examined by HPLC for signs of interaction or degradation. Excipients were chosen from materials considered to potentially cover the range of material properties that were likely to be required by this project. Materials were chosen for properties such as dissolution rate i.e. from materials that are relatively soluble in aqueous media to totally insoluble materials; their potential as viscosity/release rate modifiers, including such materials as different HPMC (viscosity) grades and Aerosils for contributing thixotropic properties. Mixes containing 25% w/w tramadol HCL were prepared for each excipient. Samples were prepared by mixing tramadol HCl with the melted excipient or for non melting excipients materials were placed in contact by blending with a 50/50 mix of excipient and fractionated coconut oil. Samples of each excipient were also stored in sealed amber glass bottles at 40° C./75% RH as control samples. The project objective describes a target of 15 binary mixes, however, 25 different mixes were made during this trial to maximize the range of excipients available for formulation.

### Dissolution Testing

Initially two test formulations were prepared as noted below. The capsules for this and all other small scale capsule preparations were manufactured by the melting and mixing of the ingredients in a water bath or on a hot plate then hand filling capsules to the target weight. All capsules used were size 1 gelatin capsules.

| | | |
|---|---|---|
| Material | % w/w | Quantity per cap mg |

Formulation 052/014

| | | |
|---|---|---|
| Poloxamer 188 | 62.8 | 282.7 |
| HPMC K100M | 17.9 | 80.3 |
| Aerosol COK 84 | 2.7 | 12.0 |
| Tramadol HCl | 16.6 | 74.9 |
| Capsule fill weight | | 450 |

Formulation 052/015

| | | |
|---|---|---|
| Gelucire 50/02 | 58.3 | 233.3 |
| HPMC Pharmacoat 606 | 19.9 | 79.8 |
| Aerosil COK 84 | 3.0 | 12.0 |
| Tramadol HCl | 18.7 | 74.9 |
| Capsule fill weight | | 400 |

The target fill weight was set as 400 mg for a size 1 capsule. Formulation 052/014 was initially targeted on a 400 mg fill, however, the mix proved too viscous to fill. Additional poloxamer 188 had to be added to reduce the mix viscosity to a level that could be encapsulated. The addition of extra poloxamer 188 required that the fill weight be increased to 450 mg. This quantity could be hand filled into a capsule and would meet the requirements of this preliminary trial, however, such a quantity would be excessive for machine filling into a size 1 capsule.

The tramadol HCl dissolution release profile was determined, for each formulation. Full dissolution testing is carried out using six individual capsule sets. Preliminary screening trials used between two and six capsules per test. This permitted several candidate formulations to be screened at once and clearly unsuitable formulations eliminated quickly. Potentially useful formulations could be modified further first before going on to six capsule sample dissolution testing.

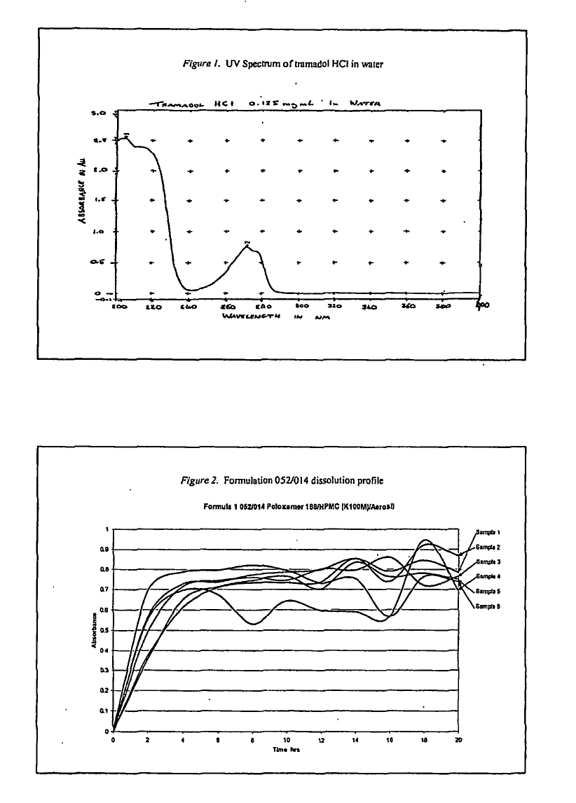

Tramadol HCl in aqueous solution shows an absorbance maximum between 240 nm and 290 nm with the maximum at 271 nm. It starts to show increasingly strong absorbance below the minimum at 240 nm to 200 nm (the limit of the instrument) however absorbance in this area is shown by many compounds so observation in the more definitive region of 240 nm to 290 was selected with 270-272 nm chosen as the preferential wavelength of observation. A plot of the UV spectrum of tramadol HCl in water is shown in FIG. 1.

Dissolution testing was carried out using the USP paddle method on a Caleva 9ST dissolution apparatus with an ERWEKA P, thermostatically controlled, water heater. Each solution was continuously cycled through a Unicam UV2-400 spectrophotometer using a Watson Marlow 205U peristaltic pump and the solution absorbance in a 1 cm silica cell, at 271 nm, recorded against the absorbance of a placebo or SIF blank with the data captured by Thermo Electron Vision UV/visible spectrometry data acquisition software protected by Vision Security. The spectrophotometer was fitted with a six cell autochanger permitting continuous automatic recording of cell solution absorbances. The capsules were weighed down with 316 stainless steel sinking wire, wrapped round each capsule. Each solution passed through a filter as it was pumped from the dissolution bath. Except where otherwise specified, the dissolution medium was 600 ml of Simulated Intestinal Fluid (SIF) USP without the inclusion of enzyme. This dissolution set up was selected to give a final absorbance value, with full release of tramadol HCl, of not more than 1.5 absorbance units (au). Typically, the final absorbance of a test solution did not exceed 1.0 au. A placebo blank was used in the reference cell. This comprised of a capsule containing the same proportion and quantity of each material used in the active test capsules but without the tramadol HCl. This ensured that the reference solution contained the same quantity (and thus gave the same background absorbance) as the excipients in the active capsules.

### Binary Mix Compatibility Study

Different materials were tested for compatibility with Tramadol HCl. The results of storage in sealed amber glass bottles under conditions of 40° C./75% RH for four weeks then subsequent analysis by HPLC for degradants or impurities are as below.

| | | | Peaks from | Impurities/Degradants | |
|---|---|---|---|---|---|
| | Material | Assay % | stressed excipient | % area normalised | Comments |
| 1 | Gelucire 44/14 | 127.9 | none | none | |
| 1 | Gelucire 44/14 REPEAT SAMPLE | 71.2 | none | none | Mean 2 samples 99.5% |
| 2 | Gelucire 50/13 | 106.3 | none | none | |
| 3 | Gelucire 43/01 | | | | Not available |
| 4 | Poloxamer 188 | 101.9 | none | none | |
| 5 | Poloxamer 124 (Pluronic L44) | 98.6 | none | none | Separated suspension |
| | | | | | re-mixed before sampling |
| 6 | PEG 6000 | 96.6 | none | none | |
| 7 | PEG 400 | 100.7 | none | none | |
| 8 | Propylene glycol | 96.5 | none | none | |
| 9 | Beeswax (refined yellow) | 2.1 | none | none | Material insoluble in |
| | | | | | sample diluent |
| 10 | Starch 1500 (+Miglyol) | 97.3 | none | none | Separated suspension |
| | | | | | re-mixed before sampling |
| 11 | Cetyl alcohol 1-hexadecanol | 4.5 | none | none | Solution produced was a |
| | | | | | thick slime |
| | | | | | Very hard to take HPLC |
| | | | | | sample |
| 12 | Paraffin wax | 15.0 | none | none | Material insoluble in |
| | | | | | sample diluent |
| 13 | Miglyol (fractionated coconut oil) | 102.3 | none | none | Separated suspension |
| | | | | | re-mixed before sampling |
| 14 | HPMC Methocel K15MP (+Miglyol) | 104.0 | none | none | |
| 15 | HPMC Methocel K100MP | 98.9 | none | none | Separated of components |
| | (+Miglyol) | | | | re-mixed before sampling |
| 16 | Methocel A (+Miglyol) | 101.1 | none | none | |
| 17 | Hydrokote 112 | 104.2 | None | None | |
| 18 | Hydrokote AP5 | 101.2 | None | None | |
| 19 | Hydrokote M | 102.8 | None | none | |
| 20 | Shellac (+Miglyol) | 99.8 | Peaks at 5.065, | RT 5.057 = 0.1% - excipient | Yellow semisolid |
| | | | 10.702 and 12.491 | RT 10.436 = 0.1% | Excipient insoluble in |
| | | | minutes | RT 10.704 = 0.5% - excipient | diluent |
| | | | | RT 12.488 = 0.3% - excipient | |
| | | | | RT 15.043 = 0.1% | |
| | | | | RT 15.402 = 0.1% | |
| 20 | Shellac UNSTRESSED | N/A | Main peaks: | N/A | Conclude: peaks present |
| | | | 5.035, 10.393, | | in stressed Shellac were |
| | | | 10.656, 12.455 | | present before stress test |
| | | | Several small peaks | | |
| | | | in time zone 14 to 18 | | |
| | | | minutes | | |
| 21 | ZeIn (+Miglyol) | 100.5 | Peak at 7.083 | RT 7.080 = 0.1% - excipient | Yellow semisolid |
| | | | minutes | | |
| 22 | Aerosil COK 84 (+Miglyol) | 100.2 | none | None | |
| 23 | Aerosil 200 (+Miglyol) | 101.9 | none | none | |
| 24 | Cithrol GMS | 99.3 | Not available | none | Solution produced a |
| | | | Control sample | | viscous mix |
| | | | 96.4% assay | | |
| 25 | Sterotex | 62.9 | none | none | Solution produced a |
| | | | | | viscous mix |
| 25 | Sterotex REPEAT SAMPLE | 32.7 | none | none | Mean 2 samples 47.8% |
| 26 | Gelucire 50/02 | 104.1 | none | none | Solution produced a |
| | | | | | viscous mix |

The results above show that none of the excipients tested show any detectable signs of degradation or interaction after one month storage under conditions of 40° C./75% RH. It was therefore possible to use any of, these materials as formulation ingredients.

### Initial Test Formulation Dissolution Testing

Preliminary test formulations were prepared based on poloxamer 188 and Gelucire 50/02. The formulation compositions are as below.

| | | |
|---|---|---|
| Material | % w/w | Quantity per cap mg |

Formulation 052/014

| | | |
|---|---|---|
| Poloxamer 188 | 62.8 | 282.7 |
| HPMC K100M | 17.9 | 80.3 |
| Aerosol COK 84 | 2.7 | 12.0 |
| Tramadol HCl | 16.6 | 74.9 |
| Capsule fill weight | | 450 |

Formulation 052/015

| | | |
|---|---|---|
| Gelucire 50/02 | 58.3 | 233.3 |
| HPMC Pharmacoat 606 | 19.9 | 79.8 |
| Aerosil COK 84 | 3.0 | 12.0 |
| Tramadol HCl | 18.7 | 74.9 |
| Capsule fill weight | | 400 |

Placebo for 052/014

| | | |
|---|---|---|
| Poloxamer 188 | 75.4 | 282.4 |
| HPMC K100M | 21.4 | 80.0 |
| Aerosol COK 84 | 3.2 | 12.0 |
| Capsule fill weight | | 374.4 |

Placebo for 052/015

| | | |
|---|---|---|
| Gelucire 50/02 | 71.5 | 232.2 |
| HPMC Pharmacoat 606 | 24.8 | 80.6 |
| Aerosil COK 84 | 3.7 | 12.1 |
| Capsule fill weight | | 325 |

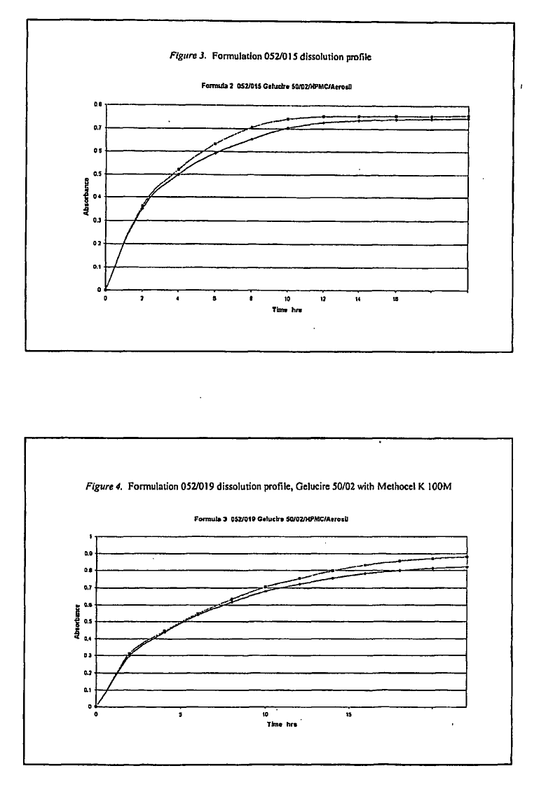

The release profiles, determined from dissolution testing in SIF are shown in FIGS. 2 and 3. Some HPMC gel remained at the end of the trial in sample 052/014 (poloxamer 188 based) but all poloxamer 188 and tramadol HCl had dissolved very quickly. Plot 2 shows that release took place over a 2-hr time span. This release rate is too fast to be useable in this project so the use of poloxamer 188 as a base excipient was discarded. The material of formulation 052/015 remained as a plug at the end of dissolution testing. It appears that the tramadol HCl and HPMC dissolved and migrates out through the Gelucire 50/02 over a period of 10-12 hr. This is shorter than the project targeted release time of 18-24 hr but Gelucire 50/02 was retained as a material worth testing further.

### Example 2

### Dissolution Testing of a Modified Gelucire 50/02 Formulation

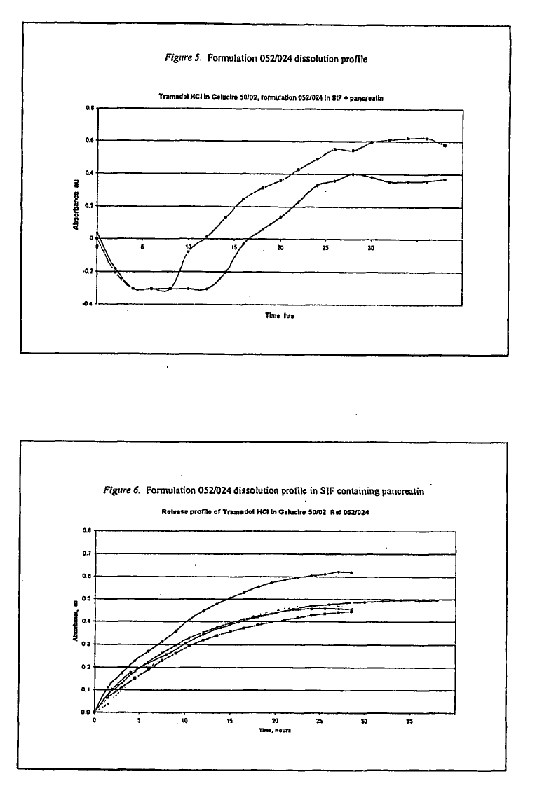

Methocel K100M, a very high viscosity HPMC, was substituted for Pharmacoat 606, a very low viscosity HPMC, to investigate whether this substitution using a much higher viscosity HPMC would significantly slow the release rate of tramadol HCl from the formulation. The active and reference placebo capsules' formulations are shown in FIG. 4. It should be noted that the relative viscosity of HPMC is based on the viscosity of a 2% aqueous solution at 20° C. measured in mPas (millipascal Seconds). The numbers and letters in the HPMC's designation indicate (different manufacturers use slightly different conventions) the HPMC's 2% viscosity in mPas (1 mPas=1 centipoise (cps)), e.g. Pharmacoat 606 (Pharmacoat 6 is the HPMC type with the final 6 referring to the 2% viscosity) has a viscosity of 6 mPas (6 centipoise) as a 2% solution while Methocel K100M (Methocel K is the HPMC type and 100M is the 2% viscosity using the letter M as the convention for a multiplication factor of 1000) has a viscosity of 100,000 mPas (100 Pascal Seconds) as a 2% solution.

| | | |
|---|---|---|
| Material | % w/w | Quantity per cap mg |

Formulation 052/019

| | | |
|---|---|---|
| Gelucire 50/02 | 58.2 | 232.9 |
| Methocel K 100M | 19.9 | 79.4 |
| Aerosil COK 84 | 3.0 | 12.0 |
| Tramadol HCl | 18.7 | 75.0 |
| Capsule fill weight | | 400 |

Placebo for 052/019

| | | |
|---|---|---|
| Gelucire 50/02 | 71.6 | 232.8 |
| HPMC Pharmacoat 606 | 24.6 | 79.8 |
| Aerosil COK 84 | 3.8 | 12.4 |
| Capsule fill weight | | 325 |

The dissolution rate had been slowed down slightly compared with 052/015 from 10-12 hr to approximately 15-18 hr, however, this mix was a thick cream and was probably too viscous to machine fill as this exact formulation.

### Example 3

### Dissolution Testing of Tramadol HCl in Gelucire 50/02 Without Additional Excipients

Initial dissolution trials on formulations were performed as ‘sighting’ trials to give some idea of the range of profiles possible for 75 mg of tramadol HCl in a matrix made up to 400 mg. The two major excipients used, poloxamer 188 and Gelucire 50/02 are at opposite ends of the water solubility/dispersibility scale so would give a good indication of the range of release rates potentially available. Poloxamer 188 is readily water soluble while Gelucire 50/02 is highly lipophilic and only very slowly dispersible in water. The Gelucire 50/02 formulation 052/019 dissolution release rate, shown in FIG. 5, is close to that desired for this project. This formulation does incorporate materials which would modify (increase) the release rate so samples were prepared containing only tramadol HCl and Gelucire 50/02 to determine the slowest release rate that could be achieved with Gelucire 50/02. Samples were prepared according to the formulation below and their release rate determined.

| | | |
|---|---|---|
| Material | % w/w | Quantity per cap mg |

Formulation 052/024

| | | |
|---|---|---|
| Gelucire 50/02 | 81.2 | 325.0 |
| Tramadol HCl | 18.8 | 75.0 |
| Capsule fill weight | | 400 |

Placebo for 052/024

| | | |
|---|---|---|
| Gelucire 50/02 | 100 | 325 |

A single capsule was initially tested then a further five capsules were also tested. All the data has been incorporated into the single plot shown below. The profile with the extended time scale is that of the first capsule tested.

These experiments indicate that full release takes place in the order of 30 hr. The outlying profiles was considered to be potentially due to uneven distribution of tramadol HCl in these hand mixed preparations but it was not deemed worthwhile to investigate this further at this stage. Gelucire 50/02 melts over a range centered on 50° C. and is hard enough to be crumbled into a powder. This makes formulations susceptible to abuse (by powdering, extraction, dose dumping, snorting etc) and it would be essential to include abuse deterrent materials such as HPMC and Aerosils in the final formulation. The release rate indicated by these profiles fall within the acceptable range of release rates worthy of further consideration at this stage of the project, however, as only two materials had been examined (with one rejected) by this stage it was decided to investigate other materials before narrowing the selection of potential formulations.

### Example 4

### Dissolution Testing of Tramadol HCl in Gelucire 50/02 in SW Containing Pancreatin

The Gelucire range of materials is described as polyglycolized glycerides consisting of mono-, di- and triglycerides and of mono- and di-fatty acid esters of polyethylene glycol (PEG) with a range of HLB (hydrophilic lipophilic balance) values from 1 to 14. A material with a value of 14 is at the hydrophilic end of the scale where the material is easily water dispersible; 1 or 2 is at the other end of the scale and the material is extremely slowly water dispersible, at best.

Gelucire 50/02 (the 02 suffix shows the HLB value to be 2) is highly lipophilic and only disperses very slowly in aqueous media. These materials are potentially digestible so it is possible that a formulation that shows very slow release in vitro, in purely aqueous media such as SIF, could show dramatically faster release due to digestion, as opposed to dispersion, in vivo in the presence of enzymes.

An experiment was performed to look for any indications that the presence of an enzyme, pancreatin, modified the release rate of tramadol HCl in Gelucire 50/02. This experiment encountered difficulties as pancreatin in solution absorbs strongly over a range exceeding that of tramadol HCl's 240 nm to 290 nm band and pancreatin in suspension tended to block the solution filters.

The dissolution profile of capsules containing formulation 052/024 was recorded using UV absorbance determination. The pancreatin level was reduced to one fifth of that specified in the USP method so that solution absorbance values did not significantly exceed 1 au. The results shown below were very erratic, however, as this was intended as no more than a check on whether this family of materials (atypical of future excipients) was susceptible to acceleration of release rate by digestion it was decided not to divert the project into the development of an HPLC assay for tramadol HCL in the presence of pancreatin at this stage.

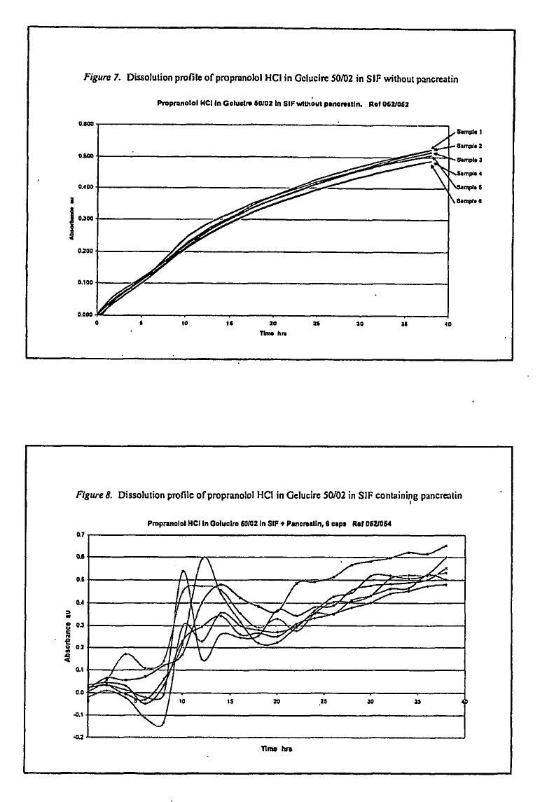

The profile (FIG. 6) shows an initial dip due to suspended/dissolved pancreatin affecting the reference cell. The absorbance of the mix appears to stop increasing after approximately 30 hr which does indicate that the tramadol HCl is fully released after this time. This corresponds well with the release time of tramadol HCl in this excipient tested in SIF in the absence of pancreatin (FIG. 5). This suggests that, at the level of pancreatin used, no major variation in dissolution release rate is observed in the presence of pancreatin. The Gelucire 50/02 units were allowed to be stirred in this medium for a further two days. The units maintained their shape and size for the entire period adding some confirmatory evidence that the Gelucire 50/02 content remained substantially unchanged (undigested).

### Example 5

### Dissolution Testing of Propranolol HCl in Gelucire 50/02 in SIF Containing Pancreatin

The above trial using Gelucire 50/02, as the base excipient, in SIP containing pancreatin suffered from the pancreatin UV absorbance overlapping and being of greater intensity than the tramadol HCl absorbance in the monitored 290 nm region. An alternative model compound was found in propranolol HCl, as a substitute for the tramadol HCl. Propranolol HCl has similar solubility and similar UV specific absorbance to tramadol HCl but has its UV absorbance maximum at 319 nm, just outside the absorbance window of pancreatin. This allowed the testing of the propranolol HCl analogue of the above formulation, 052/024, to be tested in the presence of pancreatin with reduced interference.

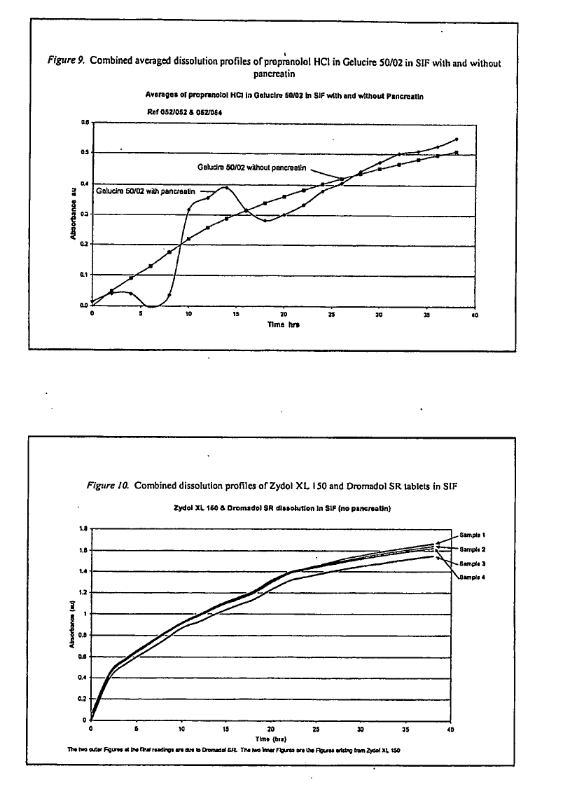

The propranolol HCl analogue was subjected to dissolution testing in 600 ml of SIF, with and without (full strength) pancreatin. Six capsule samples were tested in each case. FIGS. 7 and 8 shows data for dissolution with and without pancreatin while FIG. 9 shows the combined averaged data of dissolution in the absence and presence of pancreatin.

The pancreatin in suspension caused difficulties with filter blockage in both test and reference vessels leading to irregularities appearing in the data for propranolol HCl in SIF in the presence of pancreatin. Overall, despite the irregularities in the data, it is concluded that there is no difference detected in the overall rate of release for Gelucire 50/02 between dissolution in SIF in the absence or presence of pancreatin. This supports the conclusion reached for the similar experiment carried out using tramadol HCl in Gelucire 50/02.

### Example 6

### Dissolution Testing of Current Tramadol HCl Sustained Release Products

Tramadol HCl is available in commercial sustained release products. (for this purpose extended release, controlled release, modified release and sustained release are considered as having the same meaning). These products contain different doses of tramadol HCl, typically 150 mg, from the dosage unit under development in this project but it was considered useful to broaden our knowledge of such products and to obtain a dissolution release profile using our current conditions. It was also intended that proprietary products such as these were used later in this project as comparators during product tampering and extraction tests.

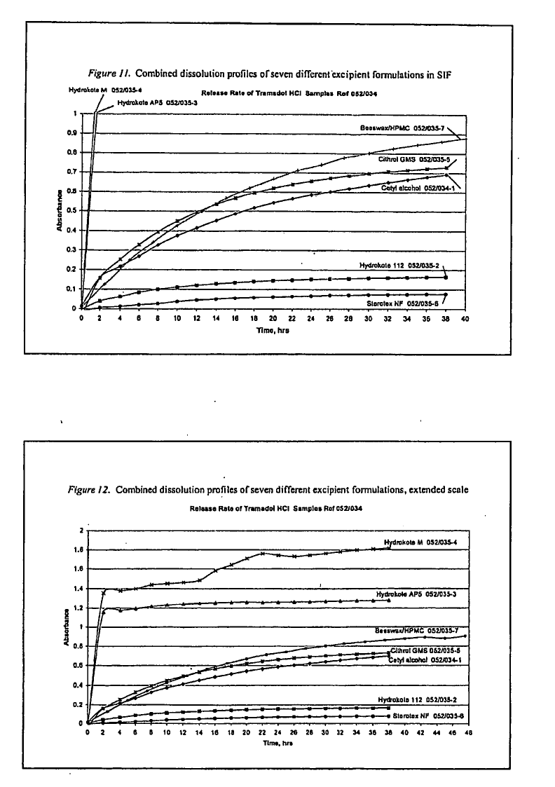

Zydol XL 150 from Pfizer for once a day administration and Dromadol SR by IVAX for twice a day administration are two proprietary products which both contain 150 mg of tramadol HCl in a sustained release formulation. Two tablets of each product had their dissolution profile determined in 600 ml of SIF without added enzyme with UV monitoring at 271 nm according to the standard method used in this development project. The combined release profiles are shown in FIG. 10. All tablets were substantially whole at the end of the test period. The release profiles match so closely that it is not possible to distinguish visually one tablet type from the other. Under the above conditions full release takes of the order of 40 hr and, as the tablets contain double the dose of the experimental formulations, the final absorbance is approximately double that shown in earlier plots. The slight dip in the plot about 17 hr is considered to be an artifact of the method.

### Example 7

### Indicative Dissolution Testing of Potential Dosage Unit Base Excipients

Previous trials demonstrated that the hard fats and slowly dissolving materials were the best choice of base material (a base excipient is the predominant excipient in a dosage unit) for a 75 mg tramadol HCl sustained release dosage unit. This identified seven other materials, from those tested in the compatibility trial, as potential base excipients. Six of these were formulated as binary mixtures with tramadol HCl and filled into capsules to a fill weight of 400 mg containing 75 mg tramadol HCl as had been carried out previously. The final material, beeswax, was formulated with the additional presence of HPMC as an unmodified formulation was unlikely to show any significant release due to the known insolubility of beeswax in aqueous media. All formulations had their dissolution profiles determined using single capsule samples for initial screening. The materials and formulations used are as below. The reference cell contained 600 mL of SIF.

| | | |
|---|---|---|
| Material | % w/w | Quantity per cap mg |

Formulation 052/034-1

| | | |
|---|---|---|
| Cetyl alcohol | 81.2 | 325.0 |
| Tramadol HCl | 18.8 | 75.0 |
| Capsule fill weight | | 400 |

Formulation 052/035-2

| | | |
|---|---|---|
| Hydrokote 112 | 81.2 | 324.8 |
| Tramadol HCl | 18.8 | 75.2 |
| Capsule fill weight | | 400 |

Formulation 052/035-3

| | | |
|---|---|---|
| Hydrokote AP5 | 81.3 | 325.2 |
| Tramadol HCl | 18.7 | 74.8 |
| Capsule fill weight | | 400 |

Formulation 052/035-4

| | | |
|---|---|---|
| Hydrokote M | 81.3 | 325.4 |
| Tramadol HCl | 18.7 | 74.6 |
| Capsule fill weight | | 400 |

Formulation 052/035-5

| | | |
|---|---|---|
| Cithrol GMS | 81.6 | 326.2 |
| Tramadol HCl | 18.4 | 73.8 |
| Capsule fill weight | | 400 |

Formulation 052/035-6

| | | |
|---|---|---|
| Sterotex NF | 81.2 | 324.9 |
| Tramadol HCl | 18.8 | 75.1 |
| Capsule fill weight | | 400 |

Formulation 052/035-7

| | | |
|---|---|---|
| Beeswax | 61.2 | 244.8 |
| Methocel K 100M | 20.1 | 80.5 |
| Tramadol HCl | 18.7 | 74.7 |
| Capsule fill weight | | 400 |

The above tests were carried out using only filtered SIF in the reference cell. Absorbance values obtained may be composed of two components, namely, absorbance due to tramadol HCl and absorbance due to dissolved excipient. 75 mg of Tramadol HCl in SIF gives an absorbance of 0.74 au therefore the absorbance must reach 0.7 au (allowing for inter capsule variation) before it is possible for all the tramadol HCl to have been dissolved. Absorbances significantly in excess of 0.7 au will have some contribution from excipient dissolution.

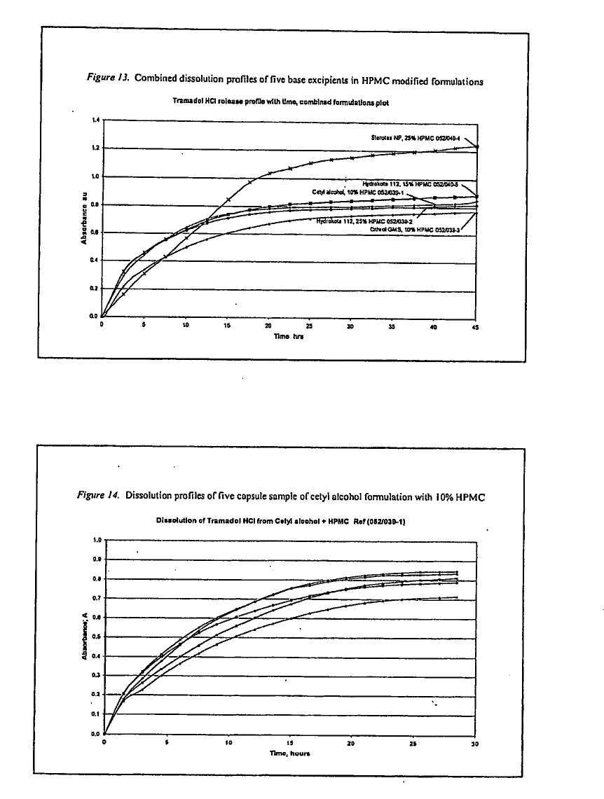

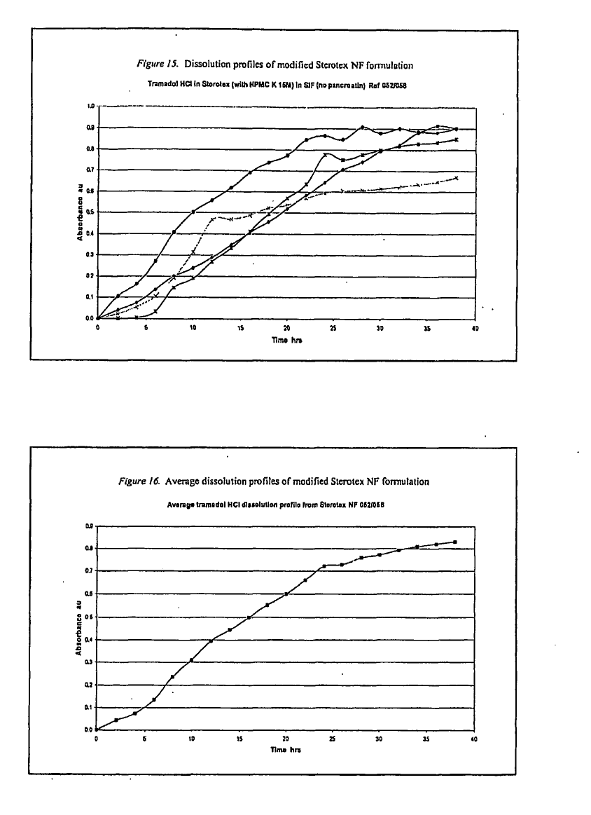

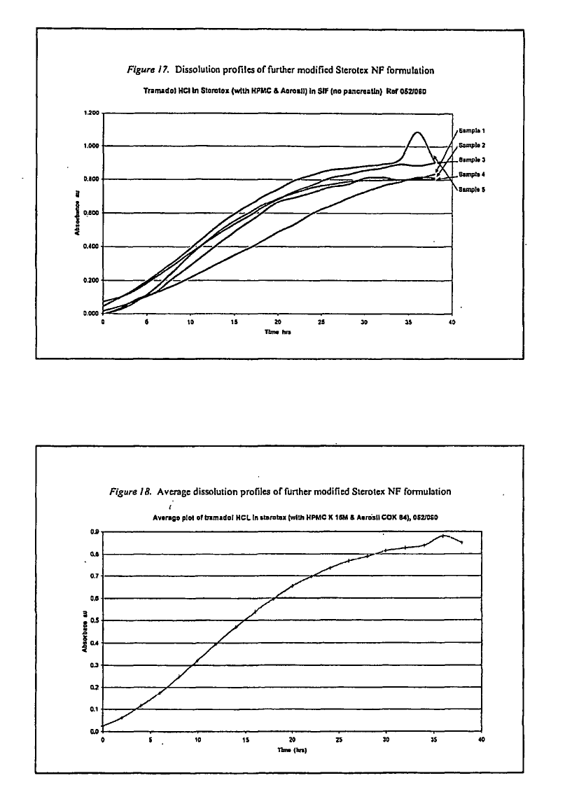

FIGS. 11 and 12 show that Hydrokote and Hydrokote AP5 dissolve rapidly and release their tramadol HCl in approximately 2 hours. This is too fast a release rate for the requirements of this project so these excipients were not able to be used as base excipients.

The other excipients were in two groups. Cithrol GMS, Cetyl alcohol and the beeswax/HPMC combination showed release rates that were slightly slower than the target of total release in 18-24 hr while the Hydrokote 112 and Sterotex NF were significantly slower. One of the requirements of this project is to develop dosage units with demonstrable deterrence to physical or solvent based tampering. Materials were to be incorporated into formulations to enhance resistance. As it was likely that these materials would accelerate release then all of the materials mentioned in this paragraph were suitable for further consideration.

### Example 8

### Dissolution Testing of Modified Tramadol HCl Formulations

The base excipients Cithrol GMS, Hydrokote 112, Cetyl alcohol, Sterotex NF and beeswax showed potential as formulation base excipients in the trial above. These materials, in binary combination (beeswax as a ternary combination), gave dissolution release rates slower than the 18-24 hr target.

In this trial HPMCs were incorporated into the formulations to accelerate release and provide a level of tamper deterrence. Up to this point formulations contained tramadol HCl, a water soluble material, with a water insoluble base excipient which could make separation by extraction relatively easy. HPMC has been chosen as a material which might enhance tamper resistance as it has the property of being water soluble and thus would ‘follow’ tramadol HCl during attempted aqueous extraction, making separation of the tramadol HCl more difficult. HPMC comes in high viscosity grades which can impart a viscous nature to aqueous extracts of dosage units i.e. if anyone tries to extract the tramadol HCl with a small amount of water in a small spoon then, at best, they will produce an unpleasant mixture with a ‘gummy’ appearance which will tend to block attempts at filtration. Additionally, HPMC behaves in an unusual manner in aqueous solution. Most water soluble materials increase in solubility as the water temperature rises. HPMC is most soluble in cold water, becoming less soluble with temperature increase until, at about 40° C., it becomes totally insoluble. Solutions of HPMC, that are heated to 40° C. or above, turn into solid gels. This means that although an HPMC may be added to increase release rates from a dosage unit, it can actively deter abuse by extraction. If an individual tries to extract tramadol HCl with warm or hot water then the HPMC will become completely insoluble and actively resist the diffusion of tramadol HCl through the relatively impermeable base excipient.

Several formulations were produced incorporating a high viscosity HPMC, Methocel K 100M, into the matrix. The formulations tested and the release profiles obtained are shown below.

| | | |
|---|---|---|
| Material | % w/w | Quantity per cap mg |

Formulation 052/039-1

| | | |
|---|---|---|
| Cetyl alcohol | 71.2 | 284.9 |
| Methocel K 100M | 10.0 | 40.0 |
| Tramadol HCl | 18.8 | 75.1 |
| Capsule fill weight | | 400 |

Formulation 052/039-2

| | | |
|---|---|---|
| Hydrokote 112 | 57.0 | 227.9 |
| Methocel K 100M | 24.5 | 97.9 |
| Tramadol HCl | 18.6 | 74.2 |
| Capsule fill weight | | 400 |

Formulation 052/040-5

| | | |
|---|---|---|
| Hydrokote 112 | 66.1 | 264.4 |
| Methocel K 100M | 15.1 | 60.3 |
| Tramadol HCl | 18.8 | 75.3 |
| Capsule fill weight | | 400 |

Formulation 052/039-3

| | | |
|---|---|---|
| Cithrol GMS | 71.0 | 284.0 |
| Methocel K 100M | 10.2 | 40.8 |
| Tramadol HCl | 18.8 | 75.2 |
| Capsule fill weight | | 400 |

Formulation 052/040-4

| | | |
|---|---|---|
| Sterotex NF | 56.5 | 225.8 |
| Methocel K 100M | 25.1 | 100.4 |
| Tramadol HCl | 18.4 | 73.8 |
| Capsule fill weight | | 400 |

FIG. 13 is based on using only SW in the reference cell. As described previously, the flattening of the curve, having reached an absorbance of at least 0.7 au, indicates full release of tramadol HCl from the dosage unit. Materials dissolving or suspending in the dissolution media may increase the recorded absorbance significantly above 0.7 as is clearly seen above for the Sterotex NF plot. FIG. 13 shows that all formulations release all/almost all tramadol HCl within approximately 17-27 hr. This is satisfactory at this stage in the project. An example of the data and scatter for a five capsule dissolution set of results produced using one of the formulations used in the combined plot above (cetyl alcohol 052/039-1) is shown in FIG. 14.

### Example 9

### Dissolution Testing of Modified Tramadol HCl in Sterotex NF Formulations

The future processing of formulations at manufacturing scale required to be considered at this stage. Some formulations had too low a viscosity, as a melt, to maintain insoluble excipients in suspension and others were so viscous that, although they could be hand filled for the purposes of these trials, they were so viscous that they would cause great difficulty during manufacture on full scale machinery. Formulations, unstable due to low viscosity, could have their viscosity increased using low levels of thixotrope but formulations of excessive viscosity required that excipients were reduced or substituted.

An Aerosil was chosen as both a thixotrope and contributor to abuse deterrence. Aerosil is the commercial name for fumed silicon dioxide manufactured by Degussa Hüls. They produce a range of Aerosils with differing properties. These include different particle size, hydrophobic or hydrophilic characteristics or blended with additional materials such as aluminium oxide for specific purposes. Aerosil COK84 was chosen as the Aerosil of choice for this project. Aerosil COK 84 is a mixture of fumed silicon dioxide and highly dispersed aluminium oxide in a 5:1 ratio. This material effectively thickens aqueous systems and other polar liquids. In this project Aerosil COK 84 will increase viscosity in a formulation, however, if attempts are made to add a small quantity of water to produce a solution (e.g. for injection) the Aerosil COK 84 will contribute to increase the viscosity of any solution produced as it is specifically designed to thicken aqueous systems. Silicon dioxide and aluminium oxide, additionally, do not melt below 100° C. (or even 1000° C.) and are insoluble. The thickening effect of this Aerosil is unaffected by heat thus an abuser attempting to melt a dosage unit will find that the structure and shape of the dosage unit tends to remain unchanged when sufficient Aerosil is incorporated even though the melting point of all other excipients has been exceeded.

Formulations were modified by having Aerosil COK 84 added in some instances to improve process characteristics and enhance abuse resistance while others had the HPMC grade substituted to bring the dissolution release rate towards the target range or to adjust the formulation properties to that required for commercial production.

The Sterotex NF formulation above, 052/040-4, contained 25% of a very high viscosity HPMC which produced a mix that could be hand filled but was excessively viscous for machine encapsulation. This formulation was modified with a lower quantity of a lower viscosity grade HPMC with the aim of producing a machine fillable formulation of similar release rate

| | | |
|---|---|---|
| Material | % w/w | Quantity per cap mg |

Formulation 052/058

| | | |
|---|---|---|
| Sterotex NF | 66.2 | 264.9 |
| Methocel K 15M | 15.0 | 60.0 |
| Tramadol HCl | 18.8 | 75.0 |
| Capsule fill weight | | 400 |

Placebo for 052/058

| | | |
|---|---|---|
| Sterotex NF | 81.5 | 265 |
| Methocel K 15M | 18.5 | 60.0 |
| Capsule fill weight | | 325 |

The dissolution profile of a four capsule sample is shown in FIGS. 15 and 16. The above profiles indicate release in 25-30 hr. (Later data will demonstrate that full release of 75 mg tramadol HCl from Sterotex NF results in an absorbance of approximately 0.8 au under the above conditions). This formulation was quite thin with fast separation of the insoluble ingredients and required an increase in viscosity. This undoubtedly contributed to the variation between individual profiles. The dosage unit was swollen after dissolution testing but retained its original shape and was tough to break up. This demonstrated that the tramadol HCl has diffused out from the dosage unit rather than released after dosage unit dissolution or disintegration.

### Example 10

### Dissolution Testing of Further Modified Tramadol HCl in Sterotex NF Formulations

Aerosil COK 84 was added to the tramadol HCl in Sterotex NF formulations. Formulations containing quantities of Aerosil COK 84 in excess of 2% w/w were too viscous for machine filling so formulation 052/058 was modified to contain 2% Aerosil COK 84 and subjected to dissolution testing against a placebo without tramadol HCl but which contained the same quantities of all other ingredients.

Formulation 052/060

| Material | % w/w | Quantity per cap mg |
|---|---|---|
| Sterotex NF | 63.9 | 255.4 |
| Methocel K 15M | 15.2 | 61.0 |
| Aerosil COK 84 | 2.1 | 8.6 |
| Tramadol HCl | 18.9 | 75.5 |
| Capsule fill weight | | 400 |

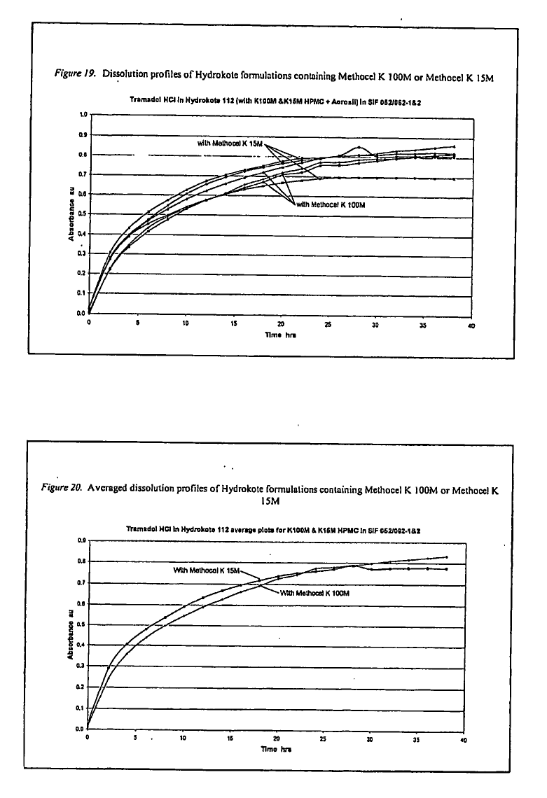

The dosage units had expanded and were soft and easily broken up after dissolution testing. The average release profile was not significantly different from that of formulation 052/058, with release in approximately 25-30 hr, however, there was less variation between individual samples indicating that low viscosity of 052/058 was a major contributor to individual sample variation (FIGS. 17 and 18).

### Example 11

### Dissolution Testing of Tramadol HCl in Hydrokote 112 with HPMC and Aerosil COK 84

FIG. 13 shows the plot for a formulation based on Hydrokote 112 containing 15% Methocel K 100M, formulation 052/040-5. Trials indicated that Aerosil COK 84 could be incorporated at 1.5% w/w to produce a flowing light cream. The above formulation was modified to contain 1.5% Aerosil COK 84 and to compare release profiles for formulations containing equal quantities of Methocel K 15M or the much higher viscosity grade Methocel K 100M. Formulations were prepared as below.

| | | |
|---|---|---|
| Material | % w/w | Quantity per cap mg |

Formulation 052/062-1

| | | |
|---|---|---|
| Hydrokote 112 | 64.7 | 258.7 |
| Methocel K 100M | 15.0 | 60.1 |
| Aerosil COK 84 | 2.1 | 8.6 |
| Tramadol HCl | 1.6 | 6.3 |
| Capsule fill weight | | 400 |

Formulation 052/062-2

| | | |
|---|---|---|
| Hydrokote 112 | 64.7 | 258.6 |
| Methocel K 15M | 15.0 | 60.2 |
| Aerosil COK 84 | 2.1 | 8.6 |
| Tramadol HCl | 1.5 | 6.2 |
| Capsule fill weight | | 400 |

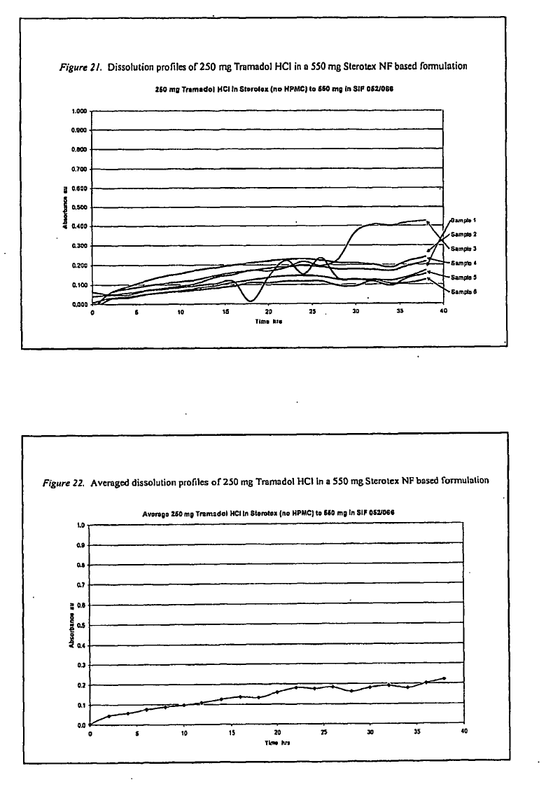

Three capsule samples of each formulation had their dissolution absorbance profiles measured in 600 mL of SIF, without enzyme at 271 nm, using the USP paddle apparatus, at 75 rpm, as carried out previously. The combined individual and averaged profiles are shown in FIGS. 19 and 20. Both dosage units were soft and crumbling at the end of dissolution testing. Both gave acceptable release times for the tramadol HCl of 25-30 hr. As would be expected, the lower viscosity grade dissolution was slightly faster than that of the formulation containing the higher viscosity grade.

### Example 12

### Dissolution Testing of a Formulation Containing 250 mg Tramadol HCl in Sterotex NF

A dosage unit containing 250 mg of tramadol HCl was considered as a future possibility for this type of slow release dosage form so a preliminary investigation was carried out to estimate the likelihood of this being achievable.

Tramadol HCl is highly water soluble. This can lead to difficulty in producing a slow release formulation as, with the preferred largest capsule size as a size 0, the largest quantity of formulated material that can be filled as a liquid fill is approximately 550 mg. This means that the formulation will contain approximately 45% as the very soluble tramadol HCl.

The objective of this exercise was to determine whether 250 mg tramadol HCl could be formulated to 500-550 mg in a mix, with the properties to enable machine filling, and having a release rate that delivered the tramadol HCl into solution over at least 18-24 hr. If the formulation released tramadol at a much slower rate then this was completely acceptable as the release rate could be accelerated by the incorporation of materials such as HPMC. Difficulties would arise if the release rate could not achieve 18-24 hr release with only the base excipient.

Sterotex NF was chosen as the base excipient for this trial as, at the 18.8% w/w tramadol HCl level (FIG. 12), it was the ‘slowest’ of the excipients under examination and able to deliver extremely slow release. A formulation targeted on 500 mg dosage was too viscous to be filled. Diluting to a total mass of 550 mg and the addition of a small quantity of Aerosil COK 84 gave a flowing cream that could be machine filled.

Formulation 052/066

| Material | % w/w | Quantity per cap mg |
|---|---|---|
| Sterotex NF | 52.8 | 290.5 |
| Aerosil COK 84 | 1.8 | 10.0 |
| Tramadol HCl | 45.4 | 249.6 |
| Capsule fill weight | | 550 |

The dissolution profile of a six capsule set was obtained in the previous manner. The only difference from previous conditions was that the dissolution medium volume had been increased to 1 liter. At this level, total release of the 250 mg of tramadol HCl would give an absorbance of at least 1.5 au. A placebo containing all materials in identical quantities without tramadol HCl was used as the reference.

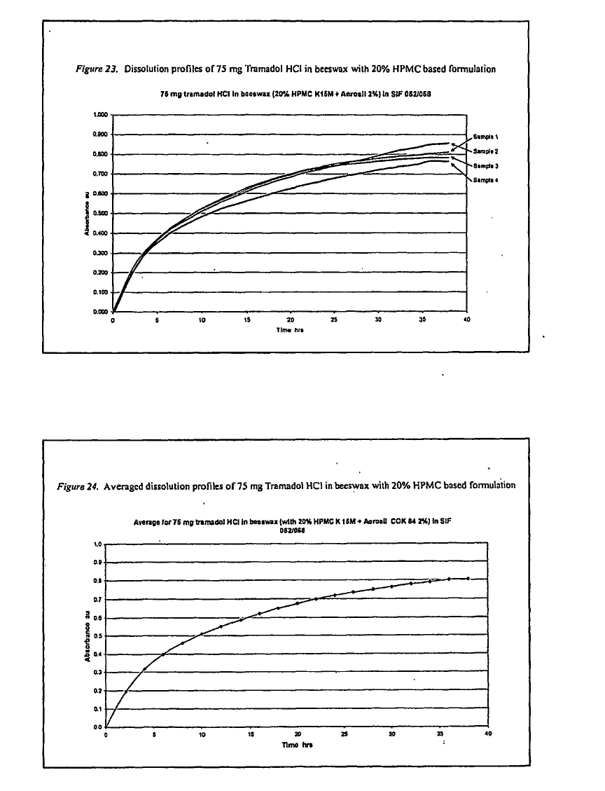

The individual plots (FIGS. 21 and 22) showed some atypical behaviour due to bubble generation in the flow through cells. Despite this, the clear observation is that this formulation released less than a quarter of its tramadol HCl content over the 38 hr period of the dissolution trial. This release time and the percentage released comfortably exceeds the minimum requirement of release of all tramadol HCl in not less than 18-24 hr. This trial demonstrates that it should be feasible to produce a similar slow release, liquid filled dosage unit to the objective of this project, containing up to 250 mg tramadol HCl in a total formulated mass of up to 550 mg.

### Example 13

### Dissolution Testing of Tramadol HCl in Beeswax Based Formulations

Previous beeswax based formulations (052/035-7), containing 20% Methocel K 100M released in a period of approximately 40 hr. This exceeded the 18-24 hr target range of the study, however, it was considered useful to include a slightly slower, in vitro, formulation to broaden the range of formulations that would eventually be subject to an in vivo trial.

Two other beeswax formulations were prepared to compare the quantity and type of HPMC that should be incorporated and the effect of Aerosil COK 84 inclusion. It was found that up to 2% Aerosil COK 84 could be included and the material remained as a potentially machine fillable mix. 25% HPMC was found to produce an excessively viscous mix. Two formulas were tested containing 20 and 23% w/w of the lower viscosity Methocel K 15M HPMC. The formulations subjected to dissolution testing were as below.

| | | |
|---|---|---|
| Material | % w/w | Quantity per cap mg |

Formulation 052/068

| | | |
|---|---|---|
| Beeswax | 59.4 | 237.6 |
| Methocel K 15M | 19.9 | 79.5 |
| Aerosil COK 84 | 2.0 | 8.2 |
| Tramadol HCl | 18.7 | 74.7 |
| Capsule fill weight | | 400 |

Formulation 052/070

| | | |
|---|---|---|
| Beeswax | 56.3 | 225.0 |
| Methocel K 15M | 23.0 | 92.0 |
| Aerosil COK 84 | 2.0 | 8.0 |
| Tramadol HCl | 18.7 | 75.0 |
| Capsule fill weight | | 400 |

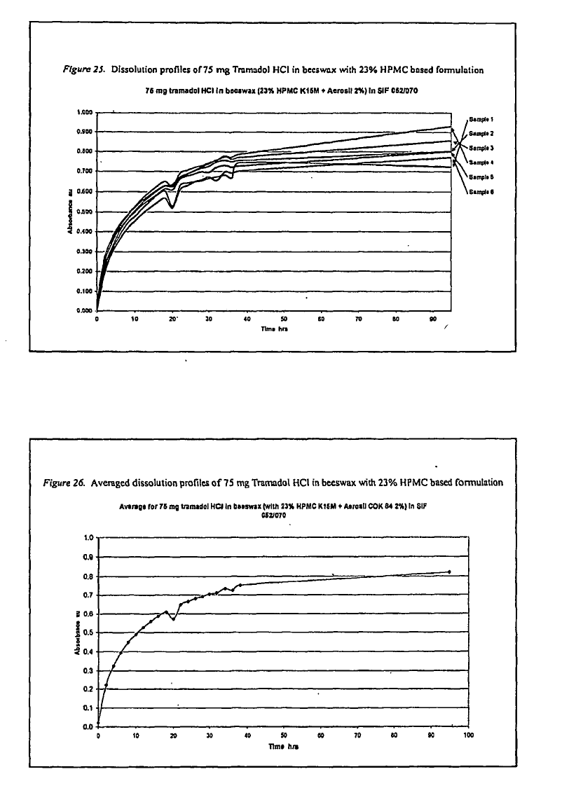

The dissolution profiles of both formulations were obtained using 600 mL of SIF and the USP paddle method with monitoring at 271 nm, unchanged from previous dissolution trials. Placebos containing all materials in identical quantities without tramadol HCl were used as the reference in each case. The dissolution profiles obtained shown in FIGS. 23, 24, 25, 26 and 27.

Tramadol HCl was released over approximately 40 hr in both cases. The dissolution of 052/070, containing 23% Methocel K 15M, was allowed to continue running for 95 hr to confirm the final absorbance achieved. It would have been expected that formulation 052/070, containing slightly more soluble matter, would have shown the faster release. It appears that there is little real difference in release rates at this level of HPMC content so the formulation containing 20% Methocel K 15M was selected for use.

### Example 14

### HPLC Analysis of Tramadol HCl During Dissolution Testing

Tramadol HCl release during dissolution testing had been monitored to this point using the absorbance of the dissolution media at 271 nm (absorbance maximum for tramadol HCl at longest wavelength) as a function of the quantity of tramadol HCl released into solution. This approach was reasonable as the excipients used in formulations were either almost insoluble or had negligible absorbance at this wavelength. It was considered that tramadol HCl was fully released when the absorbance of the solution became constant. For 75 mg tramadol formulations and the system used, this meant that the absorbance would be in excess of 0.7 au. The absorbance profile would be composed of absorbance from tramadol HCl plus a small contribution from absorbance/scattering from the other excipients.

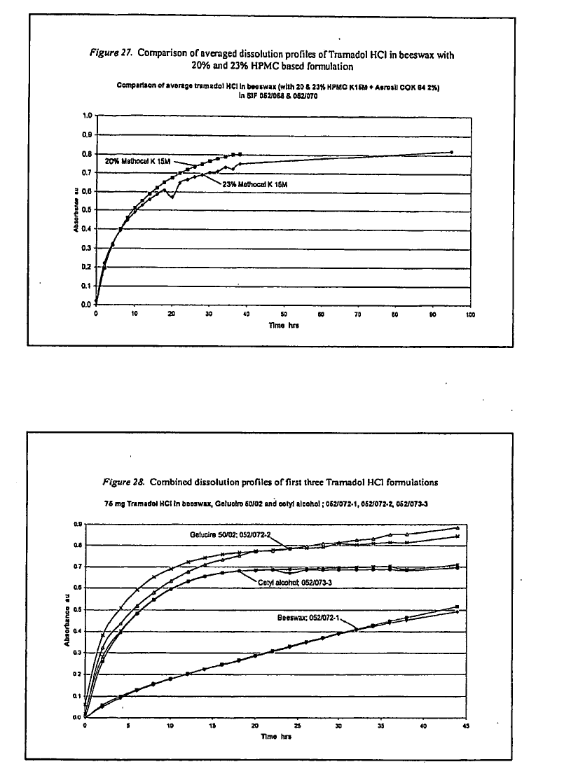

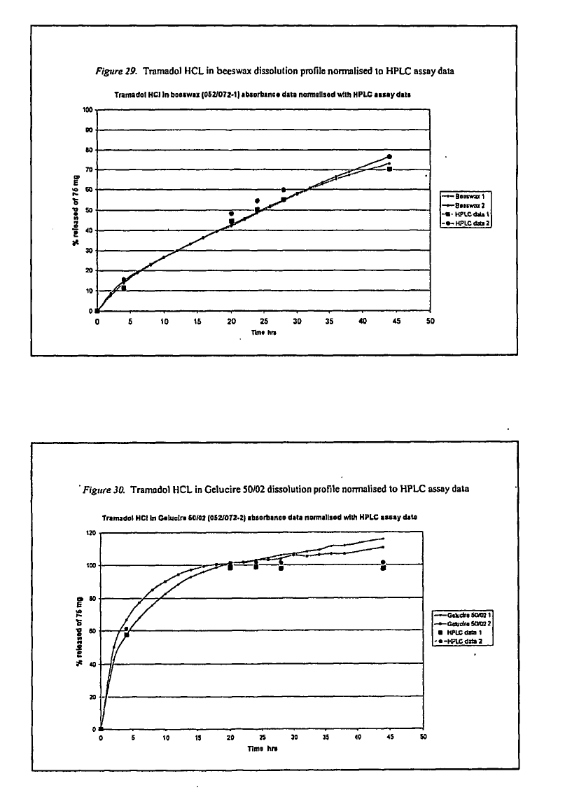

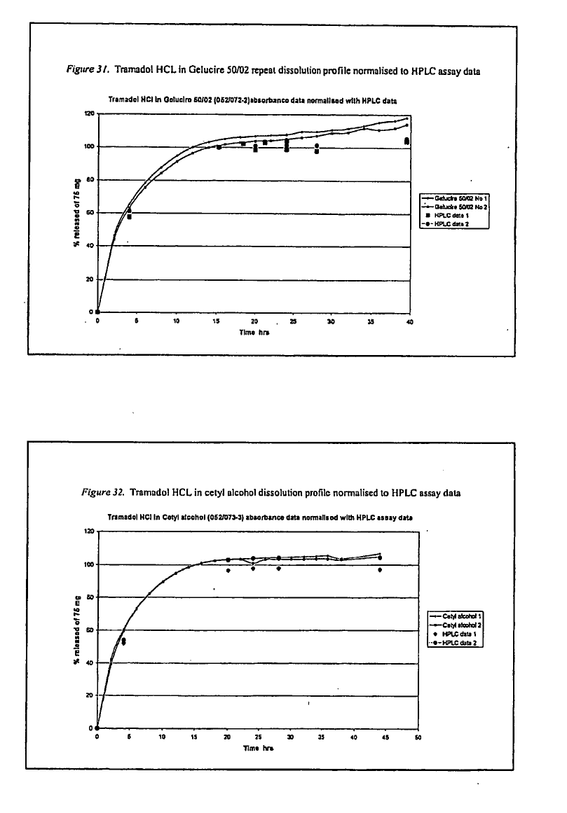

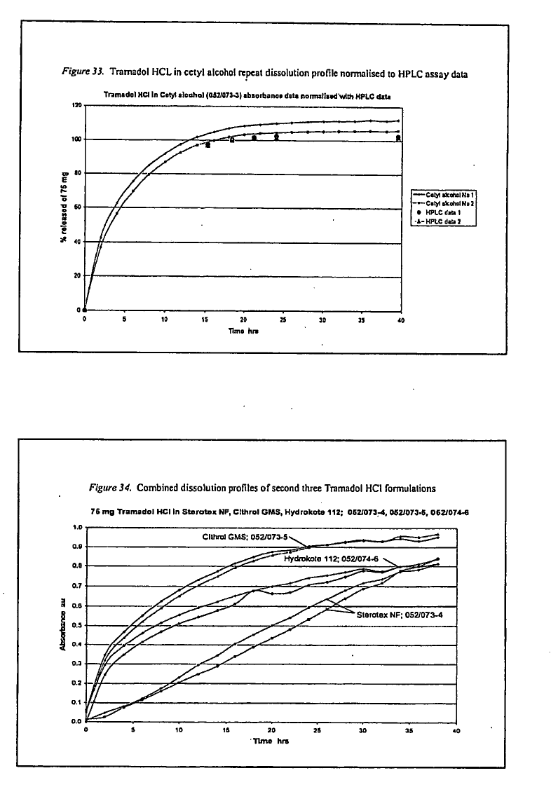

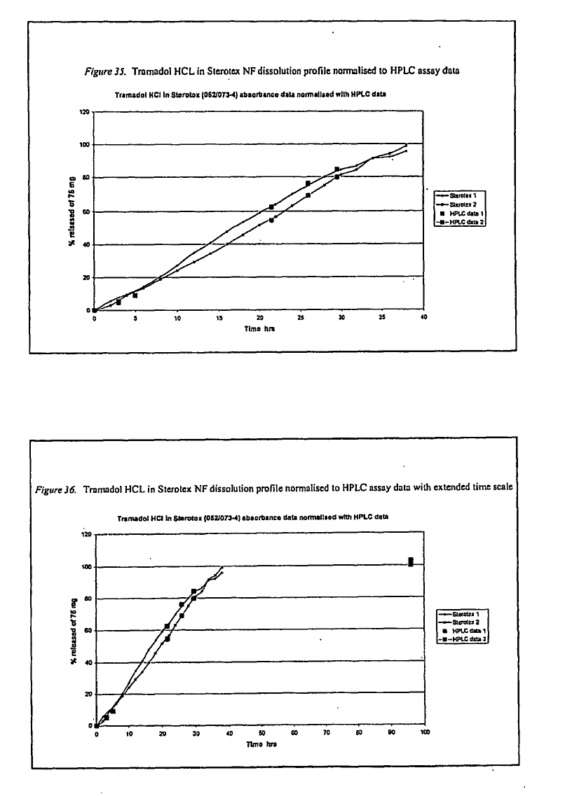

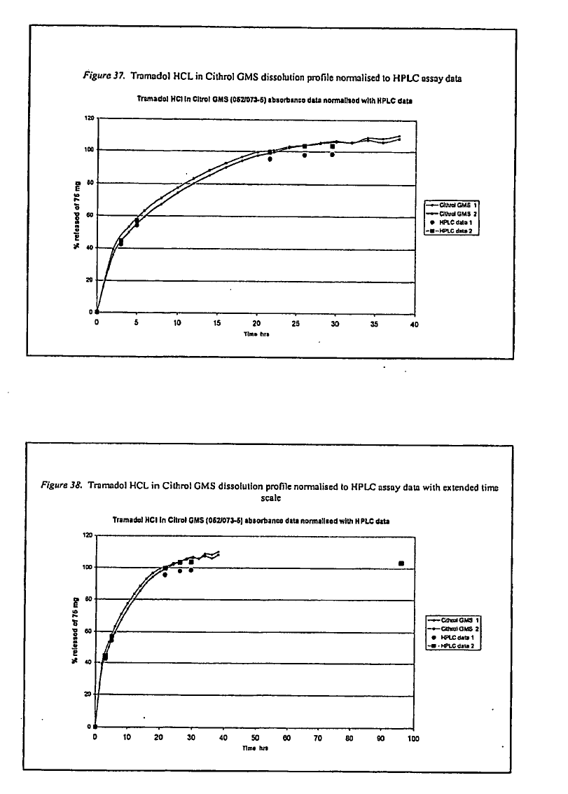

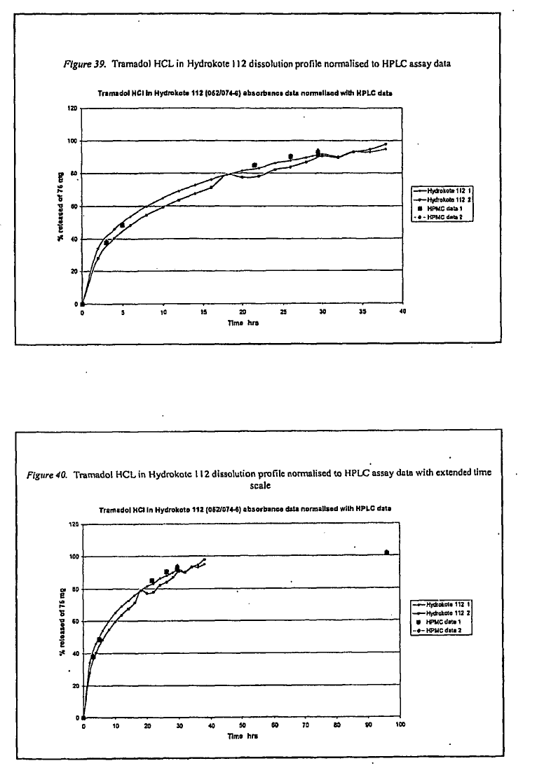

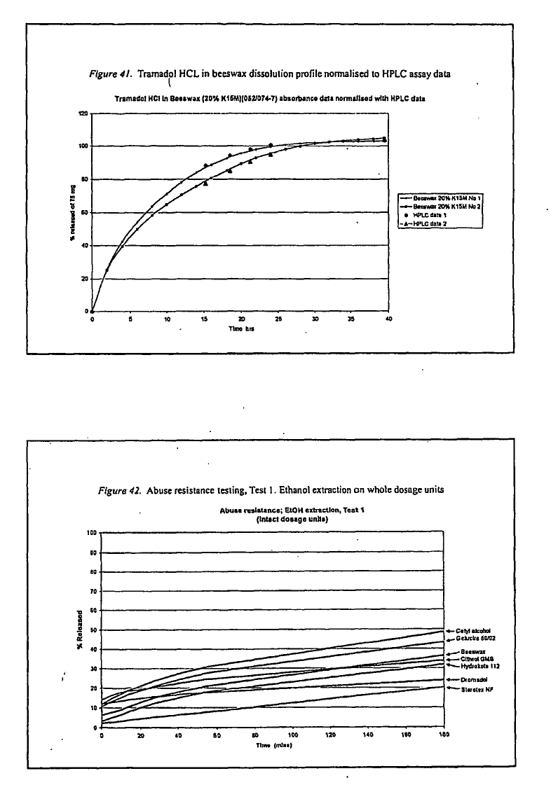

This trial subjected all of the formulations under consideration, at this point, to dissolution testing of two capsule samples (or two×two) with concurrent sampling and HPLC analysis for tramadol HCL. Sufficient samples for HPLC analysis were taken over the course of a dissolution run to allow a plot of absorbance profile versus quantity of tramadol HCl released to be constructed. This permitted the assumptions on absorbance profile versus release profile to be tested. The formulations tested are detailed below. FIG. 28 shows the combined absorbance profiles for three formulation followed by individual plots combining the percentage (of 75 mg) released into solution as determined by HPLC with the initial absorbance plot overlaid and normalised on the first or nearest position to 100% tramadol HCl release by HPLC (FIGS. 29, 30, 31, 32, 33, 34, 35, 36, 37, 38, 39, 40 and 41). This allows comparison of the quantity of tramadol HCl released and the quantity that would have been estimated from the absorbance plot as having been released. Note: The formulation reference details the exact quantities used in a particular set of samples. The same basic formula e.g. 55% of X plus 20% of Y plus 18% of Z, may appear as different formulation references as the quantities in a particular set vary slightly due to weighing variations.

| | | |
|---|---|---|
| Material | % w/w | Quantity per cap mg |

Formulation 052/072-1

| | | |
|---|---|---|
| Beeswax | 59.3 | 237.0 |
| HPMC Pharmacoat 606 | 20.0 | 79.8 |
| Aerosil COK 84 | 2.0 | 8.0 |
| Tramadol HCl | 18.8 | 75.1 |
| Capsule fill weight | | 400 |

Formulation 052/072-2 (Same as 052/019)

| | | |
|---|---|---|
| Gelucire 50/02 | 68.2 | 272.6 |
| Methocel K 100M | 10.0 | 40.1 |
| Aerosil COK 84 | 3.0 | 12.1 |
| Tramadol HCl | 18.7 | 74.8 |
| Capsule fill weight | | 400 |

Formulation 052/073-3

| | | |
|---|---|---|
| Cetyl alcohol | 67.9 | 271.5 |
| Methocel K 100M | 9.8 | 39.2 |
| Aerosil COK 84 | 3.9 | 15.8 |
| Tramadol HCl | 18.4 | 73.6 |
| Capsule fill weight | | 400 |

Formulation 052/073-4 (Similar to 052/060)

| | | |
|---|---|---|
| Sterotex NF | 64.2 | 256.8 |
| Methocel K 15M | 15.0 | 60.1 |
| Aerosil COK 84 | 2.0 | 7.9 |
| Tramadol HCl | 18.8 | 75.2 |
| Capsule fill weight | | 400 |

Formulation 052/073-5

| | | |
|---|---|---|
| Cithrol GMS | 68.3 | 273.0 |
| Methocel K 100M | 10.0 | 40.1 |
| Aerosil COK 84 | 3.0 | 12.0 |
| Tramadol HCl | 18.7 | 74.9 |
| Capsule fill weight | | 400 |

Formulation 052/074-6

| | | |
|---|---|---|
| Hydrokote 112 | 63.2 | 252.7 |
| Methocel K 15M | 15.1 | 60.2 |
| Aerosil COK 84 | 3.0 | 12.2 |
| Tramadol HCl | 18.7 | 74.9 |
| Capsule fill weight | | 400 |

Formulation 052/074-7

| | | |
|---|---|---|
| Beeswax | 59.2 | 236.9 |
| Methocel K 15M | 20.0 | 80.1 |
| Aerosil COK 84 | 2.0 | 8.1 |
| Tramadol HCl | 18.7 | 74.9 |
| Capsule fill weight | | 400 |

TABLE 2
Formulation Release Data Summary from HPLC

| | | | 100% release after |
|---|---|---|---|
| | | | approx |
| Formula | Base excipient | HPMC and % w/w | (ex HPLC data) |
| 052/072-1 | Beeswax | 20% Pharmacoat | 70-75% in 45 hr |
| | | 606 | |
| 052/072-2 | Gelucire 50/02 | 10% Methocel K | 15 hr |
| | | 100M | |
| 052/073-3 | Cetyl alcohol | 10% Methocel K | 15 hr |
| | | 100M | |
| 052/073-4 | Sterotex NF | 15% Methocel K | 38 hr |
| | | 15M | |
| 052/073-5 | Cithrol GMS | 10% Methocel K | 20 hr |
| | | 100M | |
| 052/074-6 | Hydrokote 112 | 15% Methocel K | 40 hr |
| | | 15M | |
| 052/074-7 | Beeswax | 20% Methocel K | 25 hr |
| | | 15M | |

Overall the HPLC data correlated well with absorbance data confirming that the modification of formulations based on their absorbance profiles, minimising delays that HPLC analysis would cause if applied to every sample, was a viable and acceptable approach. The above formulations cover a broad range of release profiles exceeding the 18-24 hr guide value for this project. At the present stage only the first beeswax formulation (52/072-1) is to be discontinued. Further modifications may arise during tamper resistance testing.

### Example 15

Formulations 052/074-7, 052/093-3, 052/073-5 and 052/074-6 were remanufactured with Aerosil COK 84 replaced in each with Aerosil 200. The change in Aerosil did not modify the dissolution profile or the tamper deterrence of the drug.

### Tamper Resistance Testing

The serotonin syndrome is a potentially life-threatening adverse drug experience that results from therapeutic drug use, intentional self-poisoning or inadvertent interactions between drugs. The syndrome is not an idiopathic iatrogenic reaction. Instead, it is a predictable consequence of serotonin excess in both the central and peripheral nervous systems. A wide variety of proserotonergic drugs, taken alone or in combination have been implicated in the causation of the serotonin syndrome.

Serotonin syndrome occurs with the initiation of therapy with a serotonergic agent, the addition of a second serotonergic agents and intentional or accidental overdose with one or several serotonergic agents. Serotonergic agents are frequently used in patients with primary psychopathology (major depression, schizophrenia) and in individuals with chronic pain who have comorbid depression. Such populations are particularly predisposed to concomitant therapy with multiple serotonergic drugs, other polypharmacy, drug and alcohol abuse and suicidal ideation. Consequently, patients receiving serotonergic agents are at particular risk for accidental or intentional overdose with one or several prescribed or street drugs implicated in the serotonin syndrome.

The common types of misuse of proserotonergic agents includes: 1) snorting, where the drug is inhaled as powdered dosage unit; 2) injection/ingestion (melting or extracting), where the drug is crushed and extracted or melted and the contents of a dosage unit then injects or swallows the liquid; 3) dose dumping by chewing, where the drug is chewed to increase the surface area and permit easy release of drug substance.

It is necessary to be able to measure resistance to the likely routes of abuse in a meaningful and relevant way. No standard set of tests exist with companies, interested in abuse resistance, generating their own particular set of tests. The series of tests chosen to evaluate abuse resistance and the source of the test were:

### Extraction with Alcohol on Whole Dosage Unit

This method is based on US patent application 2004/0161382 A1 (P 11, [0122]). Method: Place a whole dosage unit in 18 mL of 0.1N HCl in a 60 mL amber bottle and shake at 240 rpm on an orbital shaker for 30 min. After 30 min add 12 mL of ethanol (95-96%) to each bottle. Swirl by hand and remove a 1 mL sample from each bottle (T₀). Place the solutions back in the orbital shaker for further shaking at 240 rpm. Take 1 mL samples after 10, 20, 30, 40, 60 and 180 min of further shaking for each bottle. Analyze and graph the results on a linear scale of cumulative release (%) vs time (min).

### Extraction with Alcohol on a Crushed or Cut Dosage Unit

Extension of test in above patent. Method: Place a tablet (after crushing with a single crush with a spatula) or a capsule (cut in half) in 18 mL of 0.1N HCl in a 60 mL amber bottle and shake at 240 rpm on an orbital shaker for 30 min. Continue the test as in 1) above.

### Extraction into Water

This method is based on US patent application 2004/0161382 A1 (P12, [0130]). Method: Crush with a mortar and pestle and grind in 5 mL of water for 5 minutes. The resulting suspension is filtered through a 0.45 micron filter into a flask and diluted to 50 mL with water. Quantify Tramadol HCl concentration by HPLC.

### Freeze and Crush

Method: Freeze the dosage unit in a domestic freezer for 24 hr, then grind with a mortar and pestle for five minutes. Sieve through a suitable sieve (ca 600 micron) and, by weighing, measure the percentage passing the sieve.

### Taste of Base Excipient Mix

### Organoleptic Test

Method: Chew a placebo mix for five minutes and rate the taste on a 0-10 scale with 0 as bland to repulsive at 10. This method is relevant only to dosage units containing taste modifiers.

### Extraction into Acid

Method: Crush with a mortar and pestle and heat to boiling in 5 mL of vinegar. The resulting suspension is filtered through a 0.45 micron filter into a flask and diluted to 50 mL with water. Quantify tramadol HCl concentration by HPLC.

### Application of Heat

### Melting Temperature >50° C. or 55° C.

Method: Heat the squashed contents of a dosage unit on a hot plate until melted. Determine the temperature of melting and test whether the mix becomes sufficiently fluid to be drawn up into a syringe via a 1.2 mm needle then expelled. The formulations tested were the last six of those listed in Table 2 (omits the first sample 052/072-1). Dromadol SR tablets were included into the testing for to allow comparison of the liquid filled dosage units with a commercial tramadol HCl prolonged release preparation. The results of testing are presented below.

### Example 16

### Extraction with Alcohol on Whole Dosage Unit

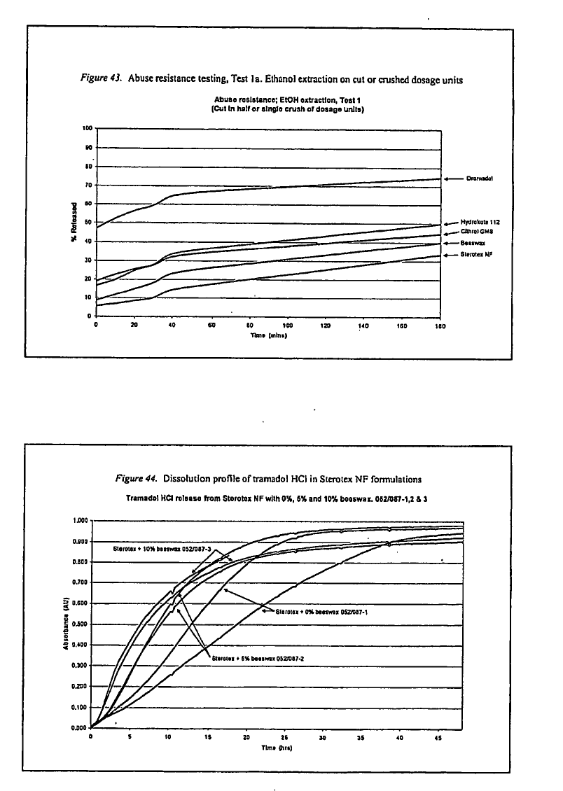

The results of this test are shown in FIG. 42.

### Example 17

### Extraction with Alcohol on Cut or Crushed Dosage Unit

The samples under test were reduced to four formulations plus the Dromadol SR comparator at this point. The Cetyl alcohol based formulation (052/073-3) and Gelucire 50/02 (052/072-2) were deselected due to their dissolution release time of approx 15 hr to 100% release and their high extractable fraction, as seen in FIG. 42. Formulations showing a slower than target in vitro release profile may possibly show more rapid release in vivo due to the presence of digestion materials but is seems unlikely that formulations showing a faster than desirable in vitro dissolution rate will show a retarded rate in vivo.

The above two tests demonstrate that whole dosage units release their contents into alcohol relatively slowly but once crushed or cut the waxy liquid fill dosage unit is much harder to extract than the tablet. One single crush turns the Dromadol tablet into an easily extractable powder. This feature would apply to any tablet. It should be noted that the apparent high quantity released at T₀ is due to the conditions specified in the method. The method requires an initial 30 min of shaking in 18 mL of 0.1N HCl before the addition of ethanol. The time is defined in the method as starting from the addition of ethanol. The tramadol HCl, shown as released at T₀, has dissolved during the 30 min pre ethanol addition sample preparation. This test demonstrates that the liquid fill formulations are clearly superior in abuse resistance by ethanol extraction to a sustained release tablet (FIG. 43).

### Example 18

### Extraction into Water Via Crushing and Grinding in Water

. The four formulations continuing under test plus Dromadol SR tablets were crushed and ground for 5 minutes in 5 mL of water to simulate extraction in preparation for swallowing or injection. The material was then filtered (by pressurising a 45μ filter using an attached syringe) and diluted before quantifying by HPLC. The results are presented in Table 3 and 4 below with comments on the mix produced after grinding given below.

TABLE 3

| Product | Observations |
|---|---|
| Dromadol SR tablet | Ground easily and formed a mobile easily |
| | filtered solution. |
| Sterotex NF formulation | Difficult to grind, forms a light paste that |
| 052/073-4 | filtered slowly. |
| Cithrol GMS formulation | Difficult to grind, forms a light paste that |
| 052/073-5 | filtered very slowly. |
| Hydrokote 112 formulation | Difficult to grind, forms a light paste that |
| 052/074-6 | filtered very slowly. |
| Beeswax formulation | Difficult to grind, forms a light paste that |
| 052/074-7 | filtered relatively easily |

The Dromadol SR tablet crushed easily and produced a solution that filtered in a matter of seconds while the beeswax formed a light paste, with difficulty, which took approximately five minutes to filter. This difficulty of preparation was common to the other capsule samples with filtration time graduating from the five minutes of the beeswax sample to over 60 minutes for the Cithrol GMS sample. All liquid fill samples gave much greater difficulty in grinding and filtering than the tablet sample.

TABLE 4
Percentage release on extraction into water.

| | | % released |
|---|---|---|
| Base excipient | Formulation | on extraction |
| Dromadol SR tablets | n/a | 84.0 |
| Sterotex NF | 052/073-4 | 38.7 |
| Cithrol GMS | 052/073-5 | 17.1 |
| Hydrokote 112 | 052/074-6 | 24.5 |
| Beeswax | 052/074-7 | 30.1 |

The HPLC data shows that tramadol HCl was easily extracted from the tablet, as would be expected as a tablet crushes easily to give a large surface area from which extraction can take place. Extraction from the liquid fill formulation was reduced considerably due to the waxy nature of the base excipients and the inclusion of HPMC which caused the liquid extracts to turn into a filtration resistant light paste.

### Example 19

### Extraction into Acid Water Via Crushing and Grinding in Dilute Acetic Acid

Dilute acetic acid (6% w/w glacial acetic in water) was used to simulate the vinegar that drug abusers may use when extracting dosage units for injection. Dosage units were crushed forcibly 2-3 times in a mortar and pestle then transferred to a small beaker where 5 mL of the above dilute acetic acid was added. The mix was heated to boiling on a hotplate and held boiling for 5-10 s. The mix was allowed to cool to room temperature, the resulting solution filtered through a 45μ filter, as above, the solution diluted to volume and the content of tramadol HCl determined by HPLC. The assay results are shown below expressed as a percentage of the contents released into solution.

TABLE 5
Percentage release on extraction into dilute acid.

| | | % released |
|---|---|---|
| Base excipient | Formulation | on extraction |
| Dromadol SR tablets | n/a | 83.9 |
| Sterotex NF | 052/073-4 | 29.3 |
| Cithrol GMS | 052/073-5 | 41.7 |
| Hydrokote 112 | 052/074-6 | 30.2 |
| Beeswax | 052/074-7 | 17.6 |

Tramadol HCl was easily extracted from the tablet. All liquid fill formulations showed appreciably better resistance to extraction. The waxy mass of the four test formulations coalesced on melting and floated as a mass on the surface. The HPMC content of the mass is insoluble above 40° C. so, instead of its normal property of assisting release at room temperature, it actively prevents release at this temperature by helping to hold the molten mass together. The tramadol HCl migrates relatively slowly to the surface when boiling agitates the mass while the powdered tablet releases most of its content instantly. It is easily understood why the formulated capsule dosages give superior extraction resistance to that of tablets.

### Example 20

### Effect of Heat on Dosage Units

Tablets can be crushed and extracted easily while soft gel contents have been known to be liquefied by slight warming (to about 40° C.) and the contents injected directly. This test records the temperature at which the meltable excipients in a formulation have liquefied and tests whether this material can be sucked into a syringe and ejected as would take place during an injection. Formulated material was placed in a beaker then slowly warmed in a water bath. The mix temperature was recorded with a calibrated thermocouple. The results are listed in Table 5 below.

TABLE 6
Melting point range and potential for direct injection

| | | | Formu- | |
|---|---|---|---|---|
| Base | Excipient | Formu- | lation | |
| excipient | mp | lation | melted | Comment |
| Sterotex NF | 61-66° C. | 052/073-4 | 65° C. | Light cream, can't suck |
| | | | | into syringe, sets |
| | | | | instantly in needle tip |
| Cithrol | 55-60° C. | 052/073-5 | 58° C. | Light cream, can't suck |
| GMS | | | | into syringe, sets |
| | | | | instantly in needle tip |
| Hydrokote | 43-46° C. | 052/074-6 | °45 C | Viscous paste, can suck |
| 112 | | | | and eject about 5 mm of |
| | | | | material from needle |
| Beeswax | 61-66° C. | 052/074-7 | 66° C. | Viscous paste, can't |
| | | | | suck into syringe, sets |
| | | | | instantly in needle tip |

All of the mixes melted around the melting points of the base excipients and, due to this elevated melting point, none could be effectively introduced into a syringe nor could be ejected (or injected).

### Example 21

### Modification to Increase Resistance to Powdering

It was observed during this trial that the Sterotex NF formulation can be powdered with careful crushing. This occurs to a lesser extent with the Cithrol GMS and Hydrokote 112 formulations. It was desirable to decrease the ease with which this formulation could be powdered. Both the Sterotex NF and Hydrokote 112 formulations gave full release of tramadol HCl in 38-40 hr during dissolution testing. It would therefore be acceptable to add modifiers that decrease the ease of crumbling formulated material into a powder even if these accelerated release. Several materials were tested including small levels of beeswax, adding hydrophilic liquids such as maltitol or glucose syrup or adding surfactants such as Crillet 4. The addition of hydrophilic liquids or surfactants immediately turned the mix into a lumpy unfillable mass by binding the powder content together. The use of these liquids was discontinued.

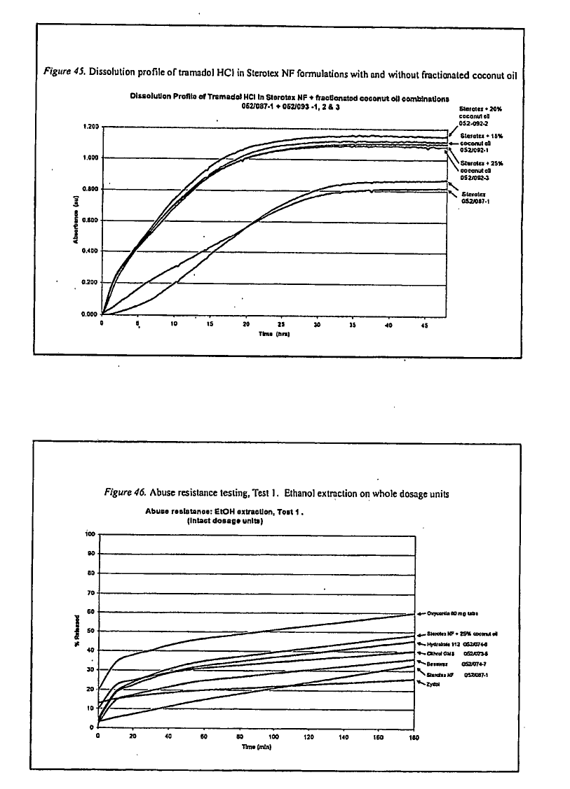

Formulations containing Sterotex NF with increased level of HPMC to accelerate dissolution plus 0, 5% and 10% beeswax were produced for examination of any change in resistance to powdering. The dissolution profiles of each formulation were recorded as the absorbance curve via UV monitoring at 271 nm as previously. The formulas used are show below. The dissolution results are show in FIG. 44.

| | | |
|---|---|---|
| Material | % w/w | Quantity per cap mg |

Formulation 052/087-1

| | | |
|---|---|---|
| Sterotex NF | 60.3 | 241.0 |
| Methocel K 15M | 20.0 | 80.0 |
| Aerosil COK 84 | 1.0 | 4.0 |
| Beeswax | 0.0 | 0.0 |
| Tramadol HCl | 18.8 | 75.0 |
| Capsule fill weight | | 400 |

Formulation 052/087-2

| | | |
|---|---|---|
| Sterotex NF | 55.3 | 221.0 |
| Methocel K 15M | 20.0 | 80.0 |
| Aerosil COK 84 | 1.0 | 4.0 |
| Beeswax | 5.0 | 20.0 |
| Tramadol HCl | 18.8 | 75.0 |
| Capsule fill weight | | 400 |

Formulation 052/087-3

| | | |
|---|---|---|
| Sterotex NF | 50.3 | 201.2 |
| Methocel K 15M | 20.0 | 79.9 |
| Aerosil COK 84 | 1.0 | 4.0 |
| Beeswax | 10.0 | 40.0 |
| Tramadol HCl | 18.8 | 74.9 |
| Capsule fill weight | | 400 |

The Sterotex formulation without beeswax showed considerable variability. The addition of 5% or 10% beeswax significantly increased the rate of release to an approximate time for full release of 25 hr. There was no meaningful difference in release rate between either formulation containing added beeswax so the formulation containing 10% beeswax (052/087-3) was selected for inclusion in subsequent trials.

### Example 22

### Ease of Powdering and Percentage of Resultant Particles of 650 Micron or Less

Capsules were initially powdered at room temperature as an indicative guide and for comparison with subsequent frozen samples. The contents were removed from the capsules and ground until the finest powder achievable had been formed. The stated period of five minutes was not normally required and it was observed that excessive grinding could cause the particles to start to coalesce. The data obtained is shown in Table 7.

TABLE 7
Powder generation by grinding of formulated material at RT

| | | | % as 650μ |
|---|---|---|---|
| Base Excipient | Formulation | Comment | or less |
| Dromadol SR | | | 64.2% |
| tablet | | | |
| Dromadol SR | | Repeat sample | 79.9% |
| tablet | | | |
| Sterotex NF | 052/087-1 | 0% beeswax | 84.7% |
| Sterotex NF | 052/087-3 | Plus 10% | 84.8% |
| | | beeswax | |
| Cithrol GMS | 052/073-5 | | 86.9% |
| Hydrokote 112 | 052/074-6 | | 2.1% |
| Beeswax | 052/074-7 | | 1.9% |

The test was repeated using capsules that had been cooled in a domestic freezer. The results of this trial are shown in Table 8.

TABLE 8
Powder generation by grinding of formulated material cooled
to domestic freezer temperatures

| | | | % as 650μ |
|---|---|---|---|
| Base Excipient | Formulation | Comment | or less |
| Dromadol SR | | | 70.6% |
| tablet | | | |
| Sterotex NF | 052/073-4 | | 78.8% |
| Sterotex NF | 052/087-3 | Plus 10% | 82.1% |
| | | beeswax | |
| Cithrol GMS | 052/073-5 | | 85.7% |
| Hydrokote 112 | 052/074-6 | | 5.5% |
| Beeswax | 052/074-7 | | 1.5% |

There was little significant difference, within experimental variation, between the results obtained at room temperature and that obtained from dosage units frozen to domestic freezer temperature (−20° C.). The Dromadol SR tablet ground to a fine powder relatively easily. The Sterotex NF and Cithrol GMS formulations also produced similar amounts of fine powder. The incorporation of 10% beeswax in one of the Sterotex NF formulations made to detectable difference. The beeswax and Hydrokote 112 formulations provided excellent resistance against powdering.

### Example 23

### Sterotex NF Formulation Modification to Enhance Resistance to Powdering

Further modifications were made to the Sterotex NF based formulation, using fractionated coconut oil, to improve resistance to powdering. Samples were prepared substituting 15, 20 and 25% of Sterotex NF for fractionated coconut oil. The formulations used were as listed below.

| | | |
|---|---|---|
| Material | % w/w | Quantity per cap mg |

Formulation 052/093-1

| | | |
|---|---|---|
| Sterotex NF | 45.2 | 180.8 |
| Fractionated coconut oil | 15.0 | 59.9 |
| Methocel K 15M | 20.0 | 80.1 |
| Aerosil COK 84 | 1.0 | 4.1 |
| Tramadol HCl | 18.8 | 75.1 |
| Capsule fill weight | | 400 |

Formulation 052/093-2

| | | |
|---|---|---|
| Sterotex NF | 40.2 | 160.8 |
| Fractionated coconut oil | 20.0 | 79.9 |
| Methocel K 15M | 20.0 | 79.9 |
| Aerosil COK 84 | 1.0 | 4.2 |
| Tramadol HCl | 18.8 | 75.1 |
| Capsule fill weight | | 400 |

Formulation 052/094-3

| | | |
|---|---|---|
| Sterotex NF | 35.3 | 141.0 |
| Fractionated coconut oil | 25.0 | 100.0 |
| Methocel K 15M | 19.9 | 79.8 |
| Aerosil COK 84 | 1.0 | 4.1 |
| Tramadol HCl | 18.8 | 75.0 |
| Capsule fill weight | | 400 |

### Example 24

The test to quantify the ease of powdering, Test 3, was repeated using capsules that had been cooled in a domestic freezer. The results of this trial are shown in table 8 below.

TABLE 9
Powder generation from Sterotex NF formulations containing fractionated
coconut oil by grinding of formulated material cooled to domestic freezer
temperatures

| | | | % as 650μ or |
|---|---|---|---|
| Base Excipient | Formulation | Comment | less |
| Sterotex NF | 052/073-4 | Data from Table 6 | 78.8% |
| Sterotex NF | 052/093-1 | Plus 15% fractionated | 49.7% |
| | | coconut oil | |
| Sterotex NF | 052/093-2 | Plus 20% fractionated | 33.7% |
| | | coconut oil | |
| Sterotex NF | 052/094-4 | Plus 25% fractionated | 8.3% |
| | | coconut oil | |

The addition of fractionated coconut oil produced the desired effect in decreasing the ability to grind cooled formulated mix into a powder. The hot mix remained a machine fillable light cream. The melting point of the 25% mix had decreased from the 65° C. melting point of a Sterotex NF mix with zero added fractionated coconut oil to an acceptable 62° C. for the mix containing 25%.

### Example 25

### Abuse Resistance Testing

### Re-Evaluation of Modified Sterotex NF Combinations

Further testing was required, after revising the Sterotex NF formulation by substituting part of the Sterotex NF for fractionated coconut oil, to determine how this change had affected the other parameters.

Dissolution testing was carried out, in the same manner as previously, using the USP paddle method to obtain the dissolution profiles of the Sterotex NF formulations with and without additional fractionated coconut oil. This plot is shown below in FIG. 45

### Example 26

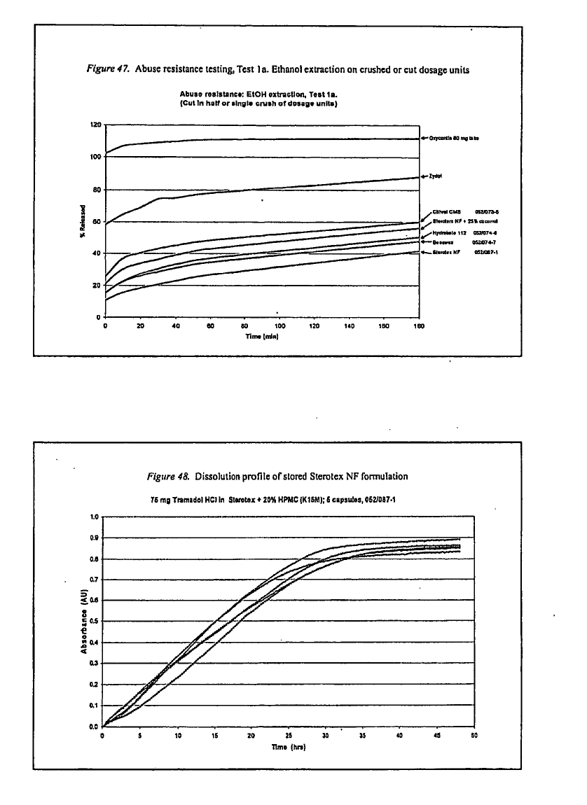

Tests for ethanol extraction of whole and crushed Or cut dosage units was also repeated. Sterotex NF with 25% fractionated coconut oil (052/094-3) was tested alongside the fractionated coconut oil free analogue (052/087-1). The opportunity was taken to test some additional relevant samples. The three previously tested formulations based on Cithrol GMS (052/073-5), Hydrokote 112 (052/074-6) and the beeswax formulation (052/074-7) were retested. Zydol XL 150 tablets were substituted for the previously used Dromadol SR tablets. Both of these are slow release formulations containing 150 mg of tramadol HCl. OxyContin extended release 80 mg tablets were included for comparison purposes as oxycodone extended release tablets are the subject of current concerns over tablet abuse and they provide another tablet comparator containing a similar quantity of water soluble active in a slow release formula. The results of ethanol extraction of whole dosage units and cut/crushed dosage units are shown below in FIGS. 46 and 47, respectively.

The Sterotex NF formulation containing 25% fractionated coconut oil did show increased susceptibility to ethanol extraction compared with the formulation without fractionated coconut oil however this was demonstrably much better than the tablets or the Cithrol GMS formulation so was considered as acceptable. The quantities extracted were broadly in line with that determined in the earlier ethanol extraction tests, shown in FIGS. 42 and 43. The Zydol XL 150 tablets showed comparable release to the Dromadol SR tablets in the earlier test. The OxyContin tablets showed much greater and faster release than any of the dosage units in either of these sets of tests.

### Example 27

The abuse resistance test involving extraction into water by grinding a dosage unit in a mortar and pestle with subsequent filtration was repeated. All of the samples included in the above ethanol extraction tests were included. Table 10 shows the results of HPLC analysis of the filtrate expressed as the percentage of drug substance released.

TABLE 10
Percentage release on extraction into water.

| | | % released |
|---|---|---|
| Base excipient | Formulation | on extraction |
| Zydol XL 150 | n/a | 87.4 |
| Oxycontin 80 mg | n/a | 90.0 |
| Sterotex NF | 052/087-1 | 28.1 |
| Sterotex NF with 25% | 052/094-3 | 11.6 |
| fr. coconut oil | | |
| Cithrol GMS | 052/073-5 | 15.3 |
| Hydrokote 112 | 052/074-6 | 23.1 |
| Beeswax | 052/074-7 | 18.6 |

### Example 28

The abuse resistance test involving extraction into dilute acetic acid by heating to boiling was repeated. The same samples as immediately above were tested and the results of HPLC analysis of the resulting filtrates are shown in table 10.

TABLE 11
Percentage release on extraction into dilute acid.

| | | % released |
|---|---|---|
| Base excipient | Formulation | on extraction |
| Zydol XL 150 | n/a | 87.4 |
| Oxycontin 80 mg | n/a | 82.2 |
| Sterotex NF | 052/087-1 | 10.8 |
| Sterotex NF with 25% | 052/094-3 | 7.0 |
| fr. coconut oil | | |
| Cithrol GMS | 052/073-5 | 34.9 |
| Hydrokote 112 | 052/074-6 | 11.1 |
| Beeswax | 052/074-7 | 14.5 |

Both sets of results gave similar results for comparable formulations in this and the earlier set of tests. All liquid fill formulations were significantly superior to any of the three commercial tablets formulations.

### Example 29

### Ease of Powdering and Percentage of Resultant Particles of 600 Micron or Less

Initial powdering tests were carried out using a laboratory stainless steel sieve of nominal 650 micron size. The sieve size used had been qualitatively determined as a size that could differentiate between the powders generated. Initially much finer sieves had been tested but were found to be too fine e.g. a 45 micron sieve was tested but this was too fine resulting in almost zero powder passing through the sieve from any samples. As result of the initial tests, a certified sieve was obtained of 600 micron size for further trials. All of the above samples were subjected to the powdering test. The results are shown in Table 12.

TABLE 12
Powder generation of formulations and comparator tablets by grinding of
dosage units cooled to domestic freezer temperatures

| | | | % as 600μ | % as 600μ |
|---|---|---|---|---|
| Base | | | or less. | or less. |
| Excipient | Formulation | Comment | Sample 1 | Sample 2 |
| Dromadol SR | n/a | | 48.1% | 51.9% |
| Zydol XL 150 | n/a | | 52.6% | 41.2% |
| Oxycontin 80 mg | n/a | | 66.6% | Not tested |
| Sterotex NF | 052/094-3 | With 25% | 2.2% | 0.6% |
| with 25% fr. | | fractionated | | |
| coconut oil | | coconut oil | | |
| Cithrol GMS | 052/073-5 | | 40.3% | 72.4% |
| Hydrokote | 052/074-6 | | 7.3% | 2.6% |
| 112 | | | | |
| Beeswax | 052/074-7 | | 0.7% | 0.6% |

It should be noted that the lower results found in this trial than those reported previously are due to a slightly finer sieve size being used. The tablets all powdered relatively easily while the Sterotex NF, Hydrokote 112 and beeswax were very resistant to powdering. The Cithrol GMS gave a high quantity of powder. The same approach of adding a room temperature oil could be used on the Cithrol GMS as used on Sterotex NF however, with the Cithrol GMS formulation showing a release rate of approximately 20 hr, on the fast size of the target 24 hr, it was decided not to amend it at this stage.

### Example 30

### Dissolution Testing of Stored Samples

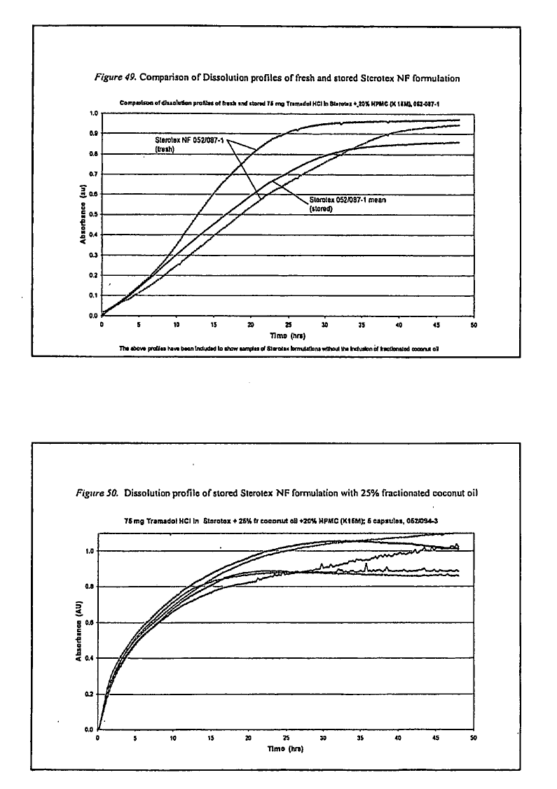

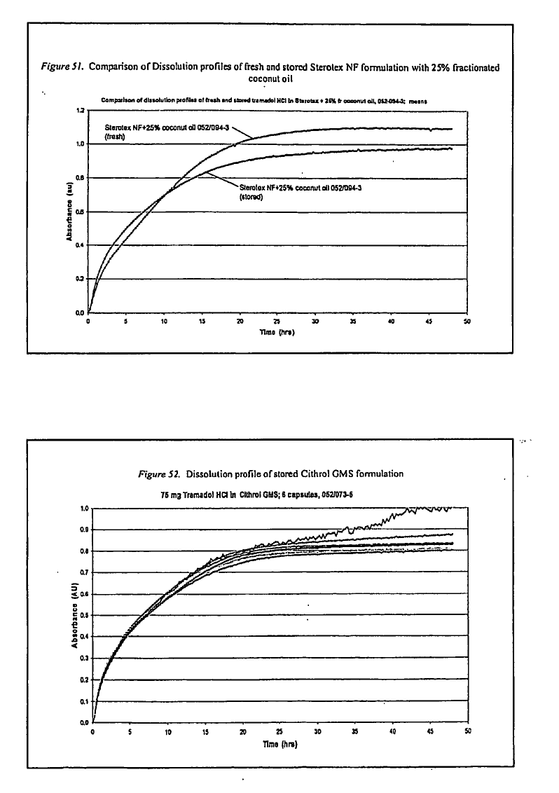

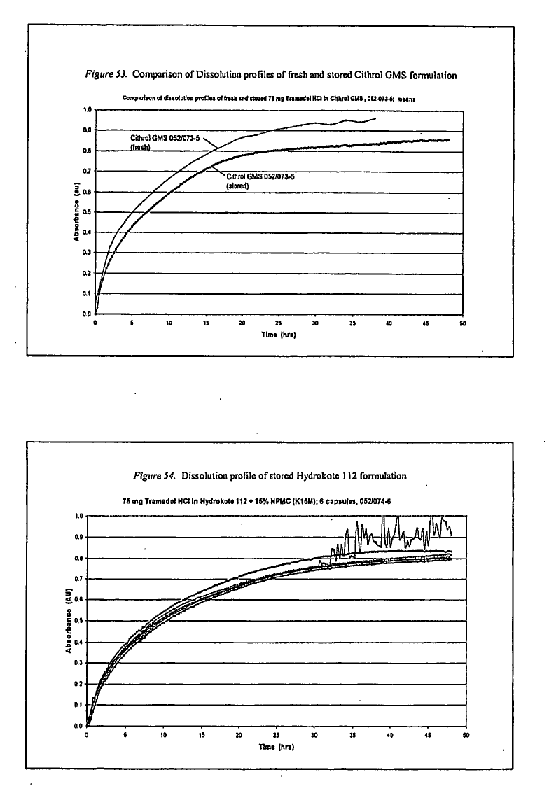

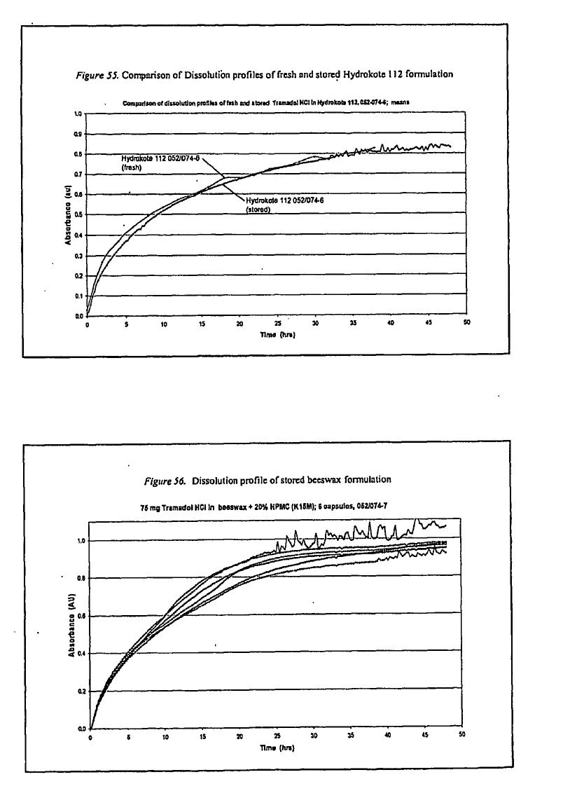

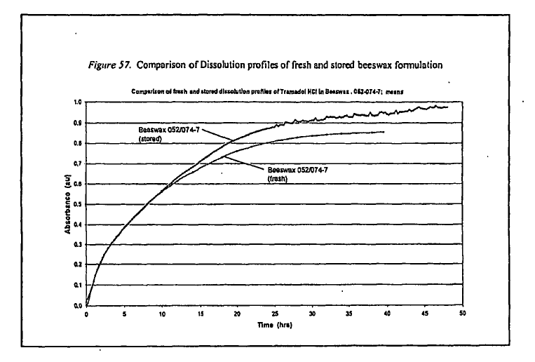

Samples of the above formulations were stored for a period of at least four weeks at room temperature (in some cases much longer) after which their dissolution release profile was redetermined. This was carried out to find out if there were any short term changes in the release rate. The tested formulations are shown in Table 13 and FIGS. 48 to 57.

TABLE 13
Formulations used for dissolution testing after a minimum of
4 weeks storage.

| | | Storage | |
|---|---|---|---|
| | | period | |
| Base Excipient | Formulation | days | Comment |
| Sterotex N | 052/087-1 | 75 | 20% HPMC |
| Sterotex NF with | 052/094-3 | 71 | |
| 25% fr. coconut oil | | | |
| Cithrol GMS | 052/073-5 | 95 | |
| Hydrokote 112 | 052/074-6 | 98 | |
| Beeswax | 052/074-7 | 83 | |

1. A method of reducing the intensity of the serotonin syndrome, the method comprising administering to a subject a proserotonergic agent and a serotonin surge protector (SSP).

2. The method of claim 1, wherein the ratio A:B is less than 10:1, A being the mean Cₘₐₓ of the proserotonergic agent following single dose oral administration of a dosage form after intentional or inadvertent tampering, and B being the mean Cₘₐₓ of the proserotonergic agent after single dose oral administration of an intact dosage form.

3. The method of claim 1, wherein the ratio C:D is less than 10:1, C being the mean Tₘₐₓ of the proserotonergic agent following single dose oral administration of a dosage form after intentional or inadvertent tampering, and D being the mean Tₘₐₓ of the proserotonergic agent after single dose oral administration of an intact dosage form.

4. The method of claim 1, wherein the ratio E:F is less than 10:1, E being the mean AUC₀₋₂ of the proserotonergic agent following single dose oral administration of a dosage form after intentional or inadvertent tampering, and F being the mean AUC₀₋₂ of the proserotonergic agent after single dose oral administration of an intact dosage form.

5. The method of claim 1, wherein the amount of said proserotonergic agent released from the dosage form based on the dissolution at 1 hour of the dosage form in 900 mL of Simulated Gastric Fluid using a USP Type II (rotating paddle method) apparatus at 50 rpm at 37 degrees ° C. is 33% or less from the intact dosage form and 50% or less from the tampered dosage form.

6. The method of claim 1, wherein the proserotonergic agent is selected from the group consisting of antidepressants, selective serotonin-reuptake inhibitors (SSRIs), selective serotonin-norepinephrine reuptake inhibitors (SNRIs), serotonin reuptake inhibitors, norepinephrine reuptake inhibitors, tricyclic, tetracyclic and non-tricyclic antidepressants, monoamine oxidase (MAO) inhibitors, antiepileptics, opioid analgesics, tramadol, antiemetics, bariatric medications, antibiotics, antivirals, and cough suppressants, given in the form of an acid, base or, optionally, in the form of a pharmaceutically acceptable salt, prodrug, ester, analog, derivative, solvate, complex, polymorph, hydrate, racemate or an individual diastereoisomers or enantiomeric isomers thereof and a mixture of these.

7. The method of claim 6, wherein the proserotonergic agent is selected from the group comprising alfentanil, allylprodine, alphaprodine, anileridine, benzylmorphine, bezitramide, buprenorphine, butorphanol, carfentanil, codeine, desmethyltramadol, dextromoramide, dezocine, dihydrocodeine, dihydromorphine, eptazocine, ethylmorphine, fentanyl, heroin, hydrocodone, hydroxymethylmorphinan, hydromorphone, hydroxypethidine, isomethadone, ketobemidone, levorphanol, levomethadone, lofentanil, meperidine, meptazinol, methadone, methylmorphine, metopon, morphine, nalbuphine, nicomorphine, norlevorphanol, normethadone, nalorphine, normorphine, oxycodone, oxymorphone, pentazocine, phenazocine, piritramide, propiram, propoxyphene, racemorphan, remifentanil, sufentanil, tapentadol, tramadol, tilidine, nor-binaltorphimine (nor-BNI), etorphine, bremazocine and ethylketocyclazocine.

8-27. (canceled)

28. The method of claim 1, wherein the SSP is selected from the group consisting of polymeric gel forming agents, nonpolymeric gel forming agents, viscosity enhancing agents, high viscosity liquids, high melting point waxes and mixtures of these.

29-49. (canceled)

50. The method of claim 1, wherein said proserotonergic agent is administered orally.

51. The method of claim 1, wherein said proserotonergic agent is administered as in immediate release form.

52. The method of claim 1, wherein said proserotonergic agent is administered in extended release form.

53. The method of claim 1, wherein the dosage form comprises at least one of acetaminophen, nitroparacectamol, a COX-2 selective non-steroidal anti-inflammatory drug, a COX-2 non-selective non-steroidal anti-inflammatory drug, a cannabinoid agonist, an opioid antagonist, a muscle relaxant, a decongestant, a hypnotic, an anxiolytic, a sedative, a laxative, caffeine, and methylphenidate.

54. A pharmaceutical composition for reducing the intensity of the serotonin syndrome, the composition comprising a pro-serotonergic agent and a serotonin surge protector (SSP).

55-108. (canceled)

109. The method of claim 1, wherein said proserotonergic agent is administered in delayed release form.

110. The method of claim 1, wherein the intensity of the serotonin syndrome is reduced sufficiently that the serotonin syndrome is prevented.
